(19) United States
(12) Patent Application Publication
(10) Pub. No.: US 2017/0027541 A1
(43) Pub. Date: Feb. 2, 2017

(54) PORTABLE ULTRASOUND CART

(71) Applicant: EDAN INSTRUMENTS, INC., Shenzhen (CN)

(72) Inventors: Richard Henderson, Sunnyvale, CA (US); Sean Murphy, Sunnyvale, CA (US)

(73) Assignee: EDAN INSTRUMENTS, INC., Shenzhen (CN)

(21) Appl. No.: 15/224,089

(22) Filed: Jul. 29, 2016

Related U.S. Application Data
(60) Provisional application No. 62/199,920, filed on Jul. 31, 2015.

(51) Int. Cl. A61B 8/00

(52) U.S. Cl. CPC A61B 8/4405; A61B 8/4427; A61B 8/546; A61B 8/461; A61B 8/565; A61B 8/467; A61B 2560/0437

(57) ABSTRACT

A portable ultrasound cart includes a support structure configured to releasably secure a portable ultrasound device. The support structure includes a wall and a planar surface which define a recess structured to receive a portion of the portable ultrasound device, a first male engagement feature positioned to engage with a first female engagement feature of the portable ultrasound device and a second male engagement feature positioned to engage with a second female engagement feature of the portable ultrasound device. The portable ultrasound cart also includes a tilt system positioned to adjust a tilt angle of the support structure relative to a remainder of the portable ultrasound cart, and a release lever linked to the second male engagement feature such that when the release lever is actuated, the second male engagement feature of the portable ultrasound cart disengages from the second female engagement feature of the portable ultrasound device.

### CROSS-REFERENCE TO RELATED APPLICATIONS

This application claims priority from U.S. Provisional Patent Application No. 62/199,920, filed on Jul. 31, 2015, the contents of which are incorporated herein by reference in their entirety.

### BACKGROUND

The present invention relates generally to the field of portable ultrasound devices. Ultrasound devices image a patient by producing and emitting ultrasonic waves with a transducer. The transducer measures returning echoes of these waves to provide data regarding the patient. The data may be analyzed and assembled into an image of the patient using a computing device. Typically, portable ultrasound devices are large systems with limited battery life. Alternatively, some portable ultrasound systems are hand-held but still relatively large. Some systems may be used with a portable ultrasound cart. It is challenging and difficult to develop an ultrasound cart which releasably secures a portable ultrasound device and allows a user to release and lift away the portable ultrasound system from the ultrasound cart with a single hand. It is further challenging and difficult to develop and ultrasound cart which automatically adjusts an angle and/or height of the portable ultrasound device.

### SUMMARY

One embodiment relates to a portable ultrasound cart which includes a support structure configured to releasably secure a portable ultrasound device. The support structure includes a wall and a planar surface which define a recess structured to receive a portion of the portable ultrasound device, a first male engagement feature positioned to engage with a first female engagement feature of the portable ultrasound device and a second male engagement feature positioned to engage with a second female engagement feature of the portable ultrasound device. The portable ultrasound cart also includes a tilt system positioned to adjust a tilt angle of the support structure relative to a remainder of the portable ultrasound cart, and a release lever linked to the second male engagement feature such that when the release lever is actuated, the second male engagement feature of the portable ultrasound cart disengages from the second female engagement feature of the portable ultrasound device. The release lever may be positioned such that the release lever may be actuated by a user at the same time and/or in the same motion as grasping a handle of the portable ultrasound device to lift the portable ultrasound device from the portable ultrasound cart. The wall and the planar surface of the portable ultrasound cart may be configured to receive a lower layer of the portable ultrasound device such that the wall obscures the lower layer when the portable ultrasound device is coupled to the portable ultrasound cart, and wherein a middle layer of the portable ultrasound device is flush with the wall of the portable ultrasound cart when the portable ultrasound device is coupled to the portable ultrasound cart.

In some embodiments, the portable ultrasound cart includes a control circuit configured to adjust the tilt angle of the support structure by controlling a tilt actuator of the tilt system based on at least one of a state of the portable ultrasound system, an identification of an operator, a previously stored tilt angle, or a received input. The portable ultrasound cart may further include a tilt control switch configured to provide an input to the control circuit, wherein the control circuit adjusts the tilt angle by controlling the tilt actuator in response to the received input from the tilt control switch. In some embodiments, the control circuit automatically adjusts the tilt angle based on an identification (ID) recognition system. For example, the control circuit may automatically adjust the tilt angle based on predefined preferences of the operator responsive to the identification of the operator, wherein the identification includes at least one of an IP address, a serial number, or a user profile of the portable ultrasound device. In further embodiments, the support structure is adjusted to a rest position responsive to at least one of the state of the portable ultrasound device being turned OFF or the portable ultrasound device being disengaged from the support structure. In still further embodiments, the portable ultrasound cart includes a plurality of user preference inputs trainable to correspond to user position preferences of different users, wherein when one of the plurality of user preference inputs is actuated, the control circuit adjusts the tilt angle by controlling the tilt actuator.

In some embodiments, the portable ultrasound cart includes a transducer housing structured to receive an ultrasound transducer, the ultrasound transducer configured to provide data to the portable ultrasound device, a rotation actuator coupled to the transducer housing and configured to rotate the transducer housing, and a control circuit configured to provide a command to the rotation actuator to rotate the transducer housing to a specific orientation based on at least one of a state of the portable ultrasound system, an identification of an operator, or a previously stored orientation. The control circuit may automatically adjust the orientation of the transducer housing based on predefined preferences of the operator responsive to the identification of the operator, wherein the identification of the operator includes at least one of an IP address, a serial number, or a user profile of the portable ultrasound device. The control circuit may cause the transducer housing to rotate to a stored orientation responsive to at least one of the portable ultrasound device being turned OFF or the portable ultrasound device being disengaged from the portable ultrasound cart. In some embodiments, the transducer housing is rotated to an operating orientation responsive to at least one of the portable ultrasound device being turned ON or the portable ultrasound device being coupled to the portable ultrasound cart.

In some embodiments, the portable ultrasound cart includes a vertical support member having a first support member and a second support member, wherein the first support member is positioned within the second support member providing a telescoping feature, the telescoping feature configured to variably adjust of a height of the portable ultrasound cart. A height actuator is configured to extend and retract the first support member with respect to the second support member, and a control circuit is configured to provide a command to the height actuator to extend or retract the first support member to a specific height based on at least one of a state of the portable ultrasound system, an identification of an operator, or a previously stored height. The control circuit may automatically adjust the height of the portable ultrasound cart based on a predefined preference of the operator responsive to the identification of the operator, wherein the identification of the operator includes at least one of an IP address, a serial number, and a user profile of a portable ultrasound device. The control circuit may automatically adjust the height of the portable ultrasound cart to a stored height responsive to at least one of the state of the portable ultrasound device being turned OFF or the portable ultrasound device being disengaged from the support structure.

Another embodiment relates to a portable ultrasound cart including a thermal management system configured to enhance thermal management capabilities of a portable ultrasound device coupled to the portable ultrasound cart. The thermal management system includes a fan configured to supplement an internal fan of the portable ultrasound device. The fan is included in a support structure configured to receive the portable ultrasound device and is positioned to increase airflow through a passageway between the portable ultrasound device and the portable ultrasound cart. The portable ultrasound device includes a bottom surface that defines a first plurality of slots and the portable ultrasound cart includes a planar surface that defines a second plurality of slots. The first plurality of slots and the second plurality of slots substantially align when the portable ultrasound device and the portable ultrasound cart are coupled together. In some embodiments, the fan is configured to force additional air through the portable ultrasound device. In alternative embodiments, the fan is configured to suck air out of exit vents included in the portable ultrasound device.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 illustrates a perspective view of a portable ultrasound device, according to one embodiment.

FIG. 2 illustrates a front view of a portable ultrasound device, according to one embodiment.

FIG. 3 illustrates a block diagram of components of a portable ultrasound device, according to one embodiment.

FIG. 4A illustrates a perspective view of a portable ultrasound system including a portable ultrasound cart and a portable ultrasound device, according to one embodiment.

FIG. 4B illustrates a block diagram of components of a portable ultrasound cart, according to one embodiment.

FIG. 5A illustrates a top view of a support structure for a portable ultrasound device, according to one embodiment.

FIG. 5B illustrates a side view of a support structure for a portable ultrasound device, according to one embodiment.

FIG. 6A illustrates a first male engagement feature of a portable ultrasound cart, according to one embodiment.

FIG. 6B illustrates a first female engagement feature of a portable ultrasound device, according to one embodiment.

FIG. 7A illustrates a second male engagement feature of a portable ultrasound cart, according to one embodiment.

FIG. 7B illustrates a second female engagement feature of a portable ultrasound device, according to one embodiment.

FIG. 8 illustrates a side view of a portable ultrasound device, according to one embodiment.

FIG. 9 illustrates a side view of a portable ultrasound system device secured to a portable ultrasound cart, according to one embodiment.

FIG. 10 illustrates a tilt feature of a portable ultrasound cart having an adjustable degree of rotation, according to one embodiment.

FIGS. 11A-11B illustrate a tilt control switch used to control a tilt feature of a portable ultrasound cart, according to one embodiment.

FIGS. 12A-12D illustrate various orientations of a transducer housing, according to one embodiment.

FIG. 13 illustrates a hand grip for a portable ultrasound cart, according to one embodiment.

FIG. 14A illustrates a thermal management system for a portable ultrasound device, according to one embodiment.

FIG. 14B illustrates a thermal management system for a portable ultrasound device, according to another embodiment.

FIG. 15A illustrates a portable ultrasound cart, according to one embodiment.

FIG. 15B illustrates a side view of a portable ultrasound cart, according to one embodiment.

FIG. 15C illustrates a rear view of a portable ultrasound cart, according to one embodiment.

FIG. 15D illustrates a top view of a portable ultrasound cart, according to one embodiment.

### DETAILED DESCRIPTION

Referring to the Figures generally, a portable ultrasound cart includes features that enhance the portability, configurability, and functionality of a portable ultrasound system. The portable ultrasound system may be used for obstetrical and gynecological imaging (e.g., measuring the size of a fetus, checking the position of a fetus, etc.), cardiac imaging (e.g., identifying abnormal heart structures, measuring blood flow, etc.), urological imaging, pulmonology examinations, imaging with abdominal sonography, and/or other ultrasound applications. The portable ultrasound cart is configured to securely support the portable ultrasound system which may be removably coupled to the portable ultrasound cart. The portable ultrasound cart may adjustably position the portable ultrasound system for use by tilting the portable ultrasound system on a support structure of the portable ultrasound cart and/or adjusting the height of the portable ultrasound system by adjusting the height of the portable ultrasound cart. This allows for the portable ultrasound cart to adjust based on ergonomic concerns and factors. The portable ultrasound cart includes one or more transducer housings which are configured to store transducers and/or ultrasound gel. In some embodiments, the transducer housings are mechanically repositionable. In some embodiments, the portable ultrasound cart may be reconfigured into a storage configuration (e.g., stored orientation) and an operating configuration (e.g., non-stored orientation). In the storage configuration, the portable ultrasound cart is compact and highly maneuverable. In the operation configuration, the portable ultrasound cart may be reconfigured to the operator's preferences. The portable ultrasound cart may include various actuators positioned to reconfigure the portable ultrasound system based on user inputs and/or predefined preferences (e.g., height, display angle, etc.). The portable ultrasound cart may also include components which support the operation of the portable ultrasound system when it is engaged with the portable ultrasound cart. For example, the portable ultrasound cart may have one or more fans and/or air channels to support cooling of the portable ultrasound system.

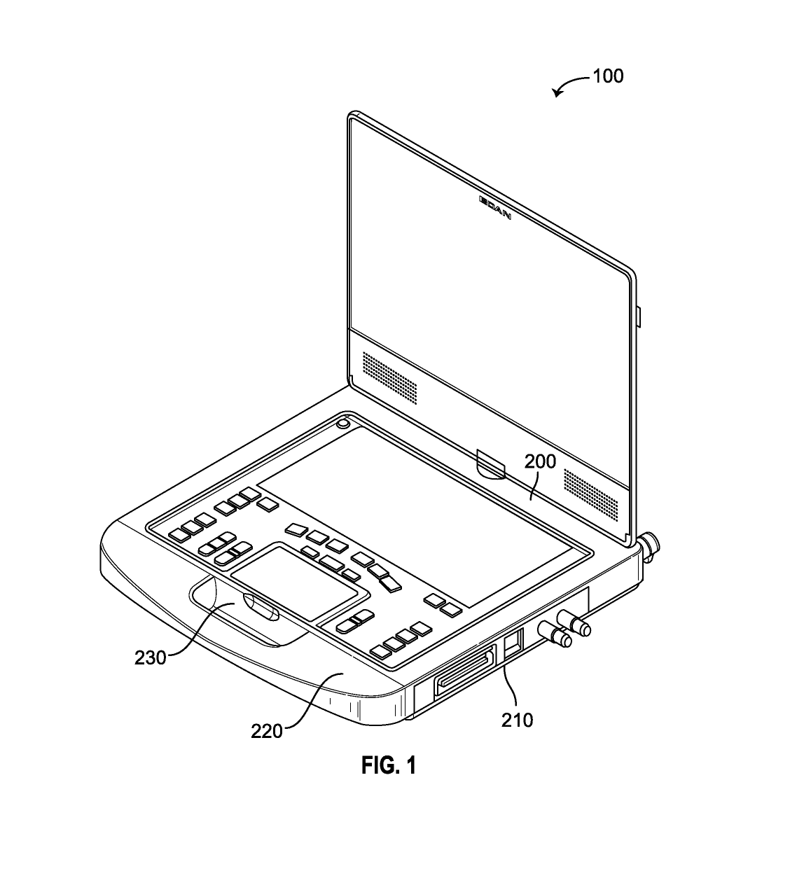

Referring to FIG. 1, one embodiment of portable ultrasound device 100 is illustrated. Portable ultrasound device 100 may include display support system 200 for increasing the durability of the display system. Portable ultrasound device 100 may further include locking lever system 210 for securing ultrasound probes and/or transducers. Some embodiments of portable ultrasound device 100 include handle system 220 for increasing portability and usability. Further embodiments include status indicator system 230 which displays, to a user, information relevant to portable ultrasound device 100. Portable ultrasound device 100 may further include features such as an easy to operate and customizable user interface, adjustable feet, a battery, one or more backup batteries, modular construction, cooling systems, etc. As shown in FIG. 1, portable ultrasound device 100 is structured as a portable device. In one embodiment, portable ultrasound device 100 is a laptop. In other embodiments, portable ultrasound device 100 is another type of portable device (e.g., a tablet, a smartphone, a form factor device, etc.).

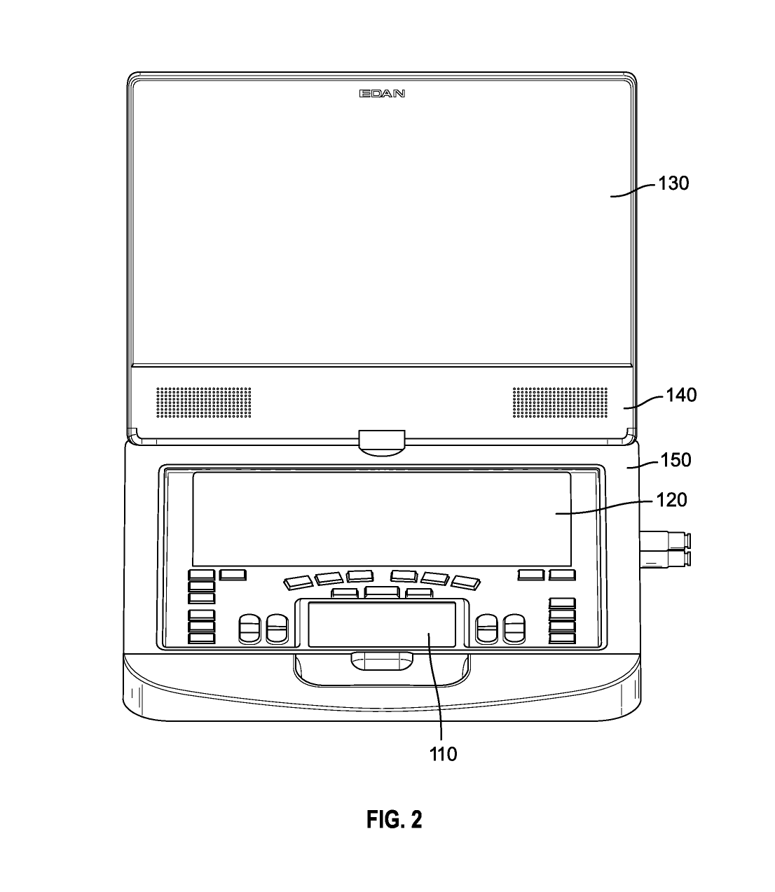

Referring to FIG. 2, a front view of one embodiment of portable ultrasound device 100 is illustrated. Main housing 150 houses components of portable ultrasound device 100. In some embodiments, the components housed within main housing 150 include locking lever system 210, handle system 220, and status indicator system 230. Main housing 150 may also be configured to support electronics modules which may be replaced and/or upgraded due to the modular construction of portable ultrasound device 100. In some embodiments, portable ultrasound device 100 includes display housing 140. Display housing 140 may include display support system 200. In some embodiments, portable ultrasound device 100 includes touchscreen 110 for receiving user inputs and displaying information, touchscreen 120 for receiving user inputs and displaying information, and main screen 130 for displaying information.

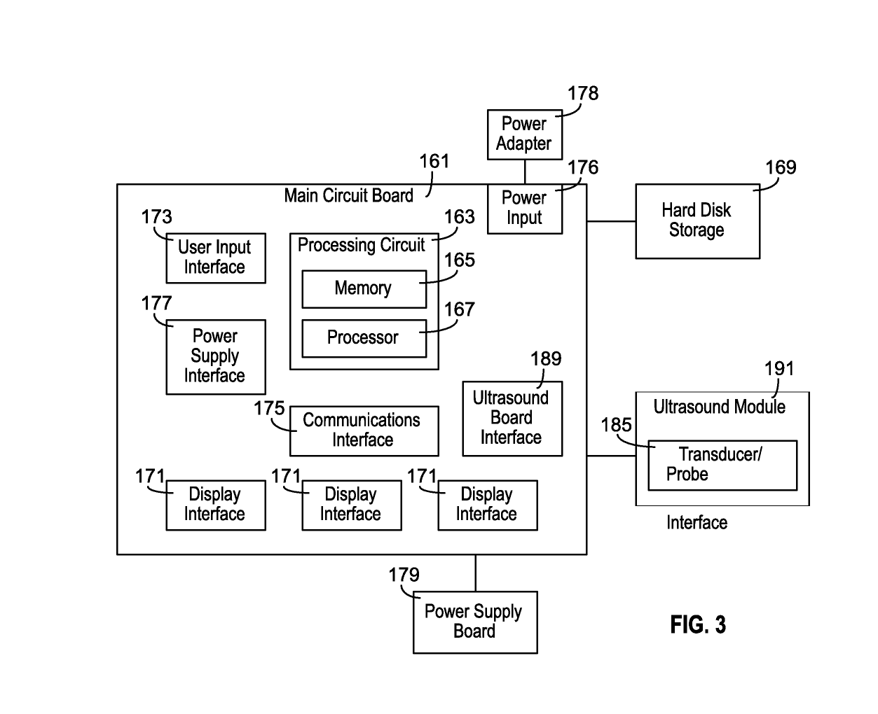

Referring to FIG. 3, a block diagram shows internal components of one embodiment of portable ultrasound device 100. Portable ultrasound device 100 includes main circuit board 161. Main circuit board 161 carries out computing tasks to support the functions of portable ultrasound device 100 and provides connection and communication between various components of portable ultrasound device 100. In some embodiments, main circuit board 161 is configured so as to be a replaceable and/or upgradable module.

To perform computational, control, and/or communication tasks, main circuit board 161 includes processing circuit 163. Processing circuit 163 is configured to perform general processing and to perform processing and computational tasks associated with specific functions of portable ultrasound device 100. For example, processing circuit 163 may perform calculations and/or operations related to producing an image from signals and or data provided by ultrasound equipment, running an operating system for portable ultrasound device 100, receiving user inputs, etc. Processing circuit 163 may include memory 165 and processor 167 for use in processing tasks. For example, processing circuit may perform calculations and/or operations.

Processor 167 may be, or may include, one or more microprocessors, application specific integrated circuits (ASICs), circuits containing one or more processing components, a group of distributed processing components, circuitry for supporting a microprocessor, or other hardware configured for processing. Processor 167 is configured to execute computer code. The computer code may be stored in memory 165 to complete and facilitate the activities described herein with respect to portable ultrasound device 100. In other embodiments, the computer code may be retrieved and provided to processor 167 from hard disk storage 169 or communications interface 175 (e.g., the computer code may be provided from a source external to main circuit board 161).

Memory 165 may be any volatile or non-volatile computer-readable storage medium capable of storing data or computer code relating to the activities described herein. For example, memory 165 may include modules which are computer code modules (e.g., executable code, object code, source code, script code, machine code, etc.) configured for execution by processor 167.

In some embodiments, main circuit board 161 includes communications interface 175. Communications interface 175 may include connections which enable communication between components of main circuit board 161 and communications hardware. For example, communications interface 175 may provide a connection between main circuit board 161 and a network device (e.g., a network card, a wireless transmitter/receiver, etc.). Communications interface 175 may further support one or more wired communications devices or ports (e.g., Universal Serial Bus (USB) ports, Firewire ports, Ethernet ports, or otherwise include hardware for wired communications. In some embodiments, communications interface 175 is built into a connector of portable ultrasound device 100 configured to removably secure portable ultrasound device 100 to portable ultrasound cart 400 (e.g., female engagement feature 507). Advantageously, communications interface 175 may provide wired or wireless communication between portable ultrasound device 100 and portable ultrasound cart 400. For example, this may allow for portable ultrasound device 100 to provide portable ultrasound cart 400 with information such as an identification of the portable ultrasound device 100 or a currently active user account or profile of portable ultrasound device 100. As described herein in more detail, this information may be used by portable ultrasound cart 400 to position portable ultrasound device 100 in a user preferred position in some embodiments. In further embodiments, a connection between communications interface 175 of portable ultrasound device 100 and portable ultrasound system interface 450 of portable ultrasound cart 400 allows for portable ultrasound cart 400 to support portable ultrasound device 100. For example, portable ultrasound cart 400 may provide imaging related computations and provide results to portable ultrasound device 100, provide electrical power to portable ultrasound device 100, or otherwise support portable ultrasound device 100.

Some embodiments of portable ultrasound device 100 include power supply board 179. Power supply board 179 includes components and circuitry for delivering power to components and devices within and/or attached to portable ultrasound device 100. In some embodiments, power supply board 179 includes components for alternating current and direct current conversion, for transforming voltage, for delivering a steady power supply, etc. These components may include transformers, capacitors, modulators, etc. to perform the above functions. Power supply board 179 may be configured to receive electrical power from a power source 440 of portable ultrasound cart 400. For example, power supply interface 177 may include circuitry to facilitate the calculation of remaining battery power, manage switching between available power sources, providing controlled power to ultrasound module 191, etc. And, power input 176 may be hardware and/or software configured to receive electrical power and provide it to one or more components of portable ultrasound device 100. For example, power input 176 may be a jack receptacle or other connector for receiving an alternating or direct current power input.

With continued reference to FIG. 3, some embodiments of main circuit board 161 include user input interface 173. User input interface 173 may include connections which enable communication between components of main circuit board 161 and user input device hardware. For example, user input interface 173 may provide a connection between main circuit board 161 and a capacitive touchscreen, resistive touchscreen, mouse, keyboard, buttons, and/or a controller for the proceeding. In one embodiment, user input interface 173 couples controllers for touchscreen 110, touchscreen 120, and main screen 130 to main circuit board 161. In other embodiments, user input interface 173 includes controller circuitry for touchscreen 110, touchscreen 120, and main screen 130. In some embodiments, main circuit board 161 includes a plurality of user input interfaces 173. For example, each user input interface 173 may be associated with a single input device (e.g., touchscreen 110, touchscreen 120, a keyboard, buttons, etc.). Some embodiments of main circuit board 161 include display interface 171. Display interface 171 may include connections which enable communication between components of main circuit board 161 and display device hardware. For example, display interface 171 may provide a connection between main circuit board 161 and a liquid crystal display, a plasma display, a cathode ray tube display, a light emitting diode display, and/or a display controller or graphics processing unit for the proceeding or other types of display hardware.

Main circuit board 161 may also include ultrasound board interface 189 which facilitates communication between ultrasound board 179 and ultrasound module 191. Ultrasound board interface 189 may include connections which enable communication between components of main circuit board 161 and ultrasound module 191. In further embodiments, ultrasound board interface 189 includes additional circuitry to support the functionality of ultrasound module 191. Ultrasound board interface 189 may include connections which facilitate use of a modular ultrasound module 191. Ultrasound module 191 may be a module (e.g., ultrasound module) capable of performing functions related to ultrasound imaging (e.g., multiplexing sensor signals from an ultrasound probe/transducer, controlling the frequency of ultrasonic waves produced by an ultrasound probe/transducer, etc.). Ultrasound module 191 may include one or more ultrasound boards containing hardware and/or software related to ultrasound imaging.

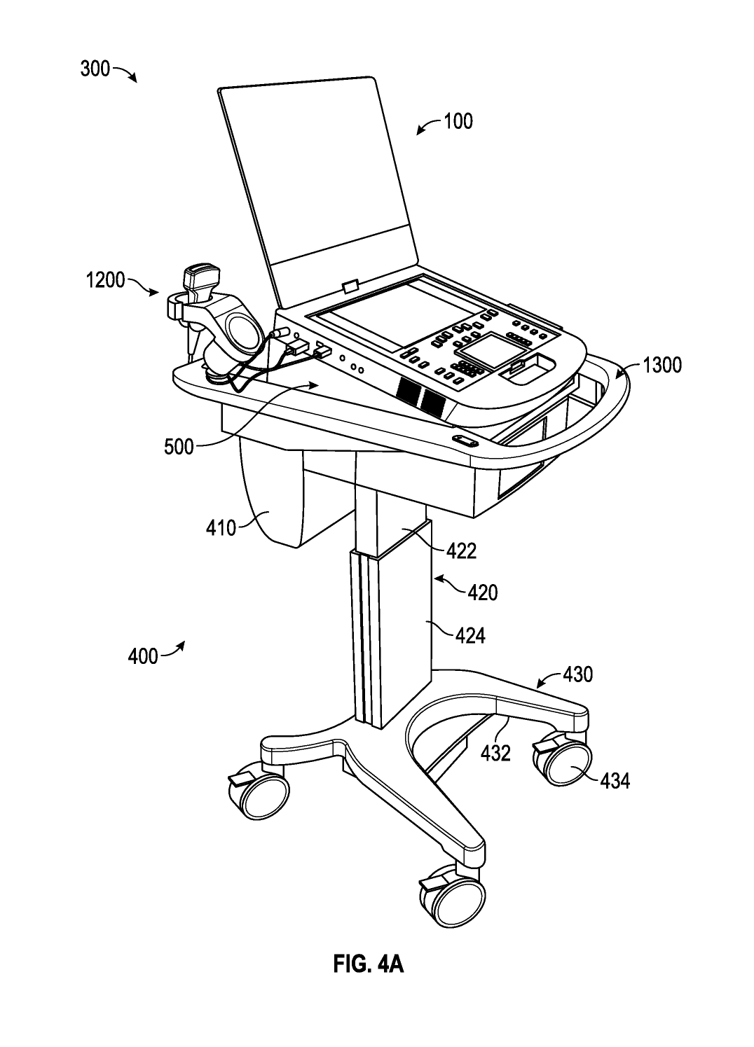

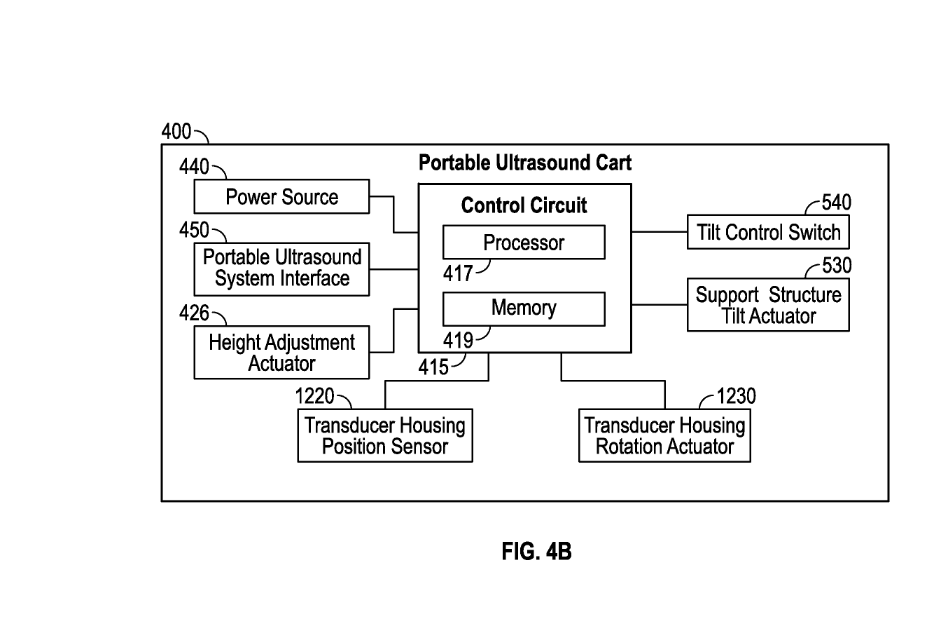

According to the example embodiments shown in FIGS. 4A-14B, portable ultrasound system 300 includes portable ultrasound device 100 and portable ultrasound cart 400. In various embodiments, portable ultrasound device 100 is configured to couple to (e.g., releasably secured to, etc.) portable ultrasound cart 400. Referring now to FIG. 4A, a perspective view of portable ultrasound system 300 is shown according to an example embodiment. As shown in FIG. 4A, portable ultrasound cart 400 includes housing 410, vertical support member 420, base member 430, support structure 500, transducer housing 1200, and handrail 1300.

Referring now to FIGS. 4A-4B, housing 410 may be structured to store various components used to operate features of portable ultrasound cart 400. For example, housing 410 may include, but not limited to, control circuit 415, power source 440, support structure tilt actuator 530, and the like. FIG. 4B illustrates a block diagram of components of one embodiment of portable ultrasound cart 400. To perform computational, control, and/or communication tasks, portable ultrasound cart 400 includes control circuit 415. Control circuit 415 is configured to control specific functions of portable ultrasound cart 400. For example, control circuit 415 may store preferred settings of an operator (e.g., a height, tilt angle, transducer housing orientation, etc.) such that when the operator interacts with portable ultrasound system 300, the control circuit 415 automatically configures portable ultrasound cart 400 to the preferred settings of the operator. Control circuit 415 may include memory 419 and processor 417 for use in processing tasks or controlling operation of portable ultrasound cart 400.

Processor 417 may be, or may include, one or more microprocessors, application specific integrated circuits (ASICs), circuits containing one or more processing components, a group of distributed processing components, circuitry for supporting a microprocessor, or other hardware configured for processing. Processor 417 is configured to execute computer code. The computer code may be stored in memory 419 to complete and facilitate the activities described herein with respect to portable ultrasound cart 400. In other embodiments, the computer code may be retrieved and provided to processor 417 from portable ultrasound system interface 450 or portable ultrasound device 100 (e.g., the computer code may be provided from a source external to control circuit 415, etc.). Memory 419 may be any volatile or non-volatile computer-readable storage medium capable of storing data or computer code relating to the activities described herein. For example, memory 419 may include modules which are computer code modules (e.g., executable code, object code, source code, script code, machine code, etc.) configured for execution by processor 417.

In some embodiments, when portable ultrasound device 100 is coupled to support structure 500, operator defined preferences or settings may be communicated to control circuit 415 via portable ultrasound system interface 450. The operator defined preferences may be determined based on a serial number or IP address of a respective portable ultrasound device 100 coupled to portable ultrasound cart 400, a user profile accessed by portable ultrasound device 100 when coupled to portable ultrasound cart 400, other unique identifier of portable ultrasound device 100 or a user thereof, or entered manually by an operator (e.g., via the portable ultrasound device 100, via buttons or levers on portable ultrasound cart 400, etc.). In some embodiments, ultrasound system interface 450 is a wireless communication transceiver (e.g., Bluetooth transceiver, near field communication transceiver, WiFi transceiver, or other wireless communications device) and control circuit 415 automatically detects and recognizes portable ultrasound device 100 when portable ultrasound device 100 comes within close proximity (e.g., communications range of ultrasound system interface 450) of portable ultrasound cart 400. In some cases, portable ultrasound device 100 automatically transmits defined preferences or settings upon establishing communication with ultrasound system interface 450 or upon receiving a request transmitted by ultrasound system interface 450 upon the establishment of communications between the two. In some cases, the portable ultrasound cart 400 and portable ultrasound device 100 may be first paired to allow for the establishment of communications automatically when the two are in communication range. For example, a Bluetooth pairing protocol may be used. In further embodiments, control circuit 415 identifies the portable ultrasound device 100 by receiving a broadcasted piece of identifying information from the portable ultrasound device and retrieves stored operator defined preferences or settings from memory 419. The operator defined preference or settings may have been previously stored in memory after being received from user input entered through an interface on portable ultrasound cart 400, received from portable ultrasound device 100, retrieved from a remote database of user preferences (e.g., stored on and/or maintained by a server), and/or received from other sources.

In some embodiments, control circuit 415 is configured to receive either identifying information used to access locally stored operator defined preferences or settings or operator defined preferences or settings directly from a radio frequency identification circuit (RFID circuit). Ultrasound system interface 450 may include or be a circuit configured to energize RFID circuits and/or receive information from RFID circuits. The RFID circuit containing the identifying information and/or the operator defined preference or settings may be a hand held tag carried by the operator of a portable ultrasound device 100 or may be incorporated into portable ultrasound device 100 such that the RFID circuit is read by ultrasound system interface 450 when portable ultrasound system 300 is in proximity to portable ultrasound cart 400 (e.g., secured to portable ultrasound cart 400). In still further embodiments, control circuit 415 is configured to receive either identifying information used to access locally stored operator defined preferences or settings or operator defined preferences or settings directly from a mobile communications device associated with a user of portable ultrasound device 100. For example, ultrasound system interface 450 may be a wireless transceiver (e.g., Bluetooth transceiver, near field communication transceiver, and/or other wireless transceiver) configured to communicate with mobile communication devices (e.g., smartphones, tablets, laptop computers, and/or other mobile communication devices). In some embodiments, the mobile communications device may be running a program or application which is configured to store operator identifying information and/or operator defined preferences or settings and provide this information wirelessly to control circuit 415 (e.g., in response to receiving a request from ultrasound system interface 450). In still further embodiments, the portable ultrasound device 100 includes a camera and facial recognition system (e.g., processor, program, and/or other components configured to perform facial recognition tasks). The portable ultrasound device 100 may detect and identify an operator of portable ultrasound device 100 based on an image including the face of the user. The portable ultrasound device 100 may then provide identifying information and/or operator preferences or settings to the portable ultrasound cart 400.

The operator defined preferences may include at least one of a tilt angle of support structure 500, a height of portable ultrasound cart 400, and an orientation of transducer housing 1200. Portable ultrasound system interface 450 is configured to facilitate communication and data transfer (e.g., via any suitable wired or wireless communications protocol, etc.) between the components of portable ultrasound cart 400 and/or portable ultrasound device 100. In some alternative embodiments, portable ultrasound cart 400 does not include portable ultrasound system interface 450 and portable ultrasound cart 400 and portable ultrasound device 100 are not in communication. In such an embodiment, control over features of portable ultrasound cart 400 (e.g., support structure tilt actuator 530, transducer housing rotation actuator 1230, height adjustment actuator 426, and/or other components) occurs based on inputs provided at portable ultrasound cart 400 from a source other than portable ultrasound device 100 (e.g., tilt control switch 540).

In one embodiment, support structure 500 of portable ultrasound cart 400 is fixed at a predetermined angle (e.g., 0 degrees, 10 degrees, 20 degrees, etc.) to provide for a better viewing of main screen 130 of portable ultrasound device 100, and easier operation of input devices of portable ultrasound device 100 (e.g., touch input devices, hard keys, or other input systems). In other embodiments, support structure 500 is configured to provide a variable display angle such that portable ultrasound device 100 may tilt through a range of angles. As shown in FIGS. 4B and 11A-11B, support structure 500 may be variably tilted by an actuator, shown as support structure tilt actuator 530, controlled via control circuit 415. Control circuit 415 may control support structure tilt actuator 530 responsive to receiving an input from an operator via a switch, shown as tilt control switch 540, or automatically based on predefined operator preferences to provide varying viewing angles for the portable ultrasound device 100. Support structure tilt actuator 530 may be an electric motor, a mechanical spring mechanism, a gas spring mechanism, or another type of actuator. In other embodiments, support structure 500 may be manually tilted by the operator (e.g., by applying a force to raise or lower support structure 500, etc.). In further embodiments, the angle of support structure 500 is changeable by both tilt actuator 530 and manually by a user. For example, a user may manually position support structure 500 at specific angle which is stored in memory 419. When powered down or otherwise not in an operational state tilt actuator 530 may move support structure 500 to an angle of 0 degrees or otherwise store support structure 500. On power up or receiving an operator identifier or ultrasound device 100 identified, control circuit 415 may read the angle stored in memory 419 and cause tilt actuator 530 to position support structure 500 at the stored angle.

As shown in FIGS. 11A and 11B, tilt control switch 540 is located on handrail 1300 in some embodiments. In other embodiments, tilt control switch 540 is located in another location. Tilt control switch 540 may include a first portion, shown as increase tilt portion 542, and a second portion, shown as decrease tilt portion 544. Increase tilt portion 542 of tilt control switch 540 is configured to send signals to control circuit 415 when pressed to actuate support structure tilt actuator 530 to facilitate an increase in the tilt angle of support structure 500. Decrease tilt portion 544 of tilt control switch 540 is configured to send signals to control circuit 415 when pressed to actuate support structure tilt actuator 530 to facilitate a decrease in the tilt angle of support structure 500. In other embodiments, tilt control switch 540 includes two switches, one configured to increase the tilt angle and the other configured to reduce the tilt angle. In some embodiments, tilt control switch 540 is a physical rocker switch, two button switched, a capacitive input switch or switches, other touch sensitive switch (e.g., resistive touch input switch), or other type of input device. In some other embodiments, the operator inputs a desired tilt angle into portable ultrasound device 100 which is communicated to control circuit 415 via portable ultrasound system interface 450. Control circuit 415 in turn actuates support structure tilt actuator 530 based on the operator input.

In an alternative embodiment, the tilt angle of support structure 500 is provided by a predefined operator preference. For example, control circuit 415 automatically provides commands to support structure tilt actuator 530 based on predefined operator preferences. The operator preferences may be stored in memory 419 and may be provided to support structure tilt actuator 530 responsive to an indication (e.g., identification, etc.) of who is operating portable ultrasound system 300 (e.g., a serial number or an IP address of a respective portable ultrasound device 100 coupled to portable ultrasound cart 400, a user profile accessed by portable ultrasound device 100 when coupled to portable ultrasound cart 400, etc.). In other embodiments, control circuit 415 automatically provides commands to support structure tilt actuator 530 based on the state of the portable ultrasound system 300 (e.g., ON, OFF, screen opened or closed, sleep mode active, hibernate mode active, etc.). Similarly, the height of portable ultrasound cart 400 may be adjusted using height adjustment actuator 426 and/or the position of transducer housings 1200 may be adjusted using rotation actuator 1230 based on operator preferences.

In still further embodiments, portable ultrasound cart 400 stores the previous tilt angle in memory 419. When portable ultrasound device 100 is powered off or removed from portable ultrasound cart 400 (e.g., as sensed by a reed switch, pressure transducer, or other switch or sensor configured to determine if the portable ultrasound device 100 is on or coupled to portable ultrasound cart 400), control circuit 415 causes support structure 500 to return to a rest position (e.g., 0 degrees, 10 degrees, etc.). When portable ultrasound system 300 is placed on, coupled to, powered on, or otherwise placed in an operational state, control circuit 415 may read from memory the last tilt setting and cause support structure tilt actuator 530 to tilt support structure 500 to the angle stored in memory. Advantageously, this allows portable ultrasound cart 400 to remember previous position settings and to position portable ultrasound device 100 in both a stored or parked position and an active or operational position. This function may be provided in embodiments in which portable ultrasound device 100 and portable ultrasound cart 400 do not communicate. Similarly, the height of portable ultrasound cart 400 and/or the position of transducer housings 1200 may be adjusted using height adjustment actuator 426 based on stored settings.

In still further embodiments, portable ultrasound cart 400 includes one or more buttons which correspond to different stored user preferences for the positioning of portable ultrasound device 100. Portable ultrasound cart 400 can be trained to store position settings corresponding to each button. For example, a user may position portable ultrasound device 100 and/or transducer housings 1200 at a desired position (e.g., using tilt control switch 540, manually positioning, or otherwise positioning portable ultrasound device 100). The user may then hold a corresponding button for greater than a predetermined time period (e.g., greater than 3 seconds). In response, control circuit 415 stores the position settings in memory 419. When the button is pressed (e.g., less than the predetermined time period), control circuit 415 reads the stored position settings from memory and causes the portable ultrasound device 100 to be positioned according to the stored settings (e.g., controls tilt actuator 530, height adjustment actuator 426 and/or rotation actuator 1230).

The techniques described herein for positioning portable ultrasound device 100 and/or otherwise controlling portable ultrasound cart 400 may be used in any combination or without combination with other techniques. For example, portable ultrasound cart 400 may have a shut down or park mode and an operation mode in which the portable ultrasound device 100 is positioned between a stored position and the previous position. And, the position of portable ultrasound device 100 may be adjusted using one or more buttons on portable ultrasound cart 400 corresponding to stored user preferences. Or, the position of the portable ultrasound system in operation mode may be provided for by setting or preferences stored in memory 165 of portable ultrasound device 100 or based on an identification of portable ultrasound device 100 or a user thereof.

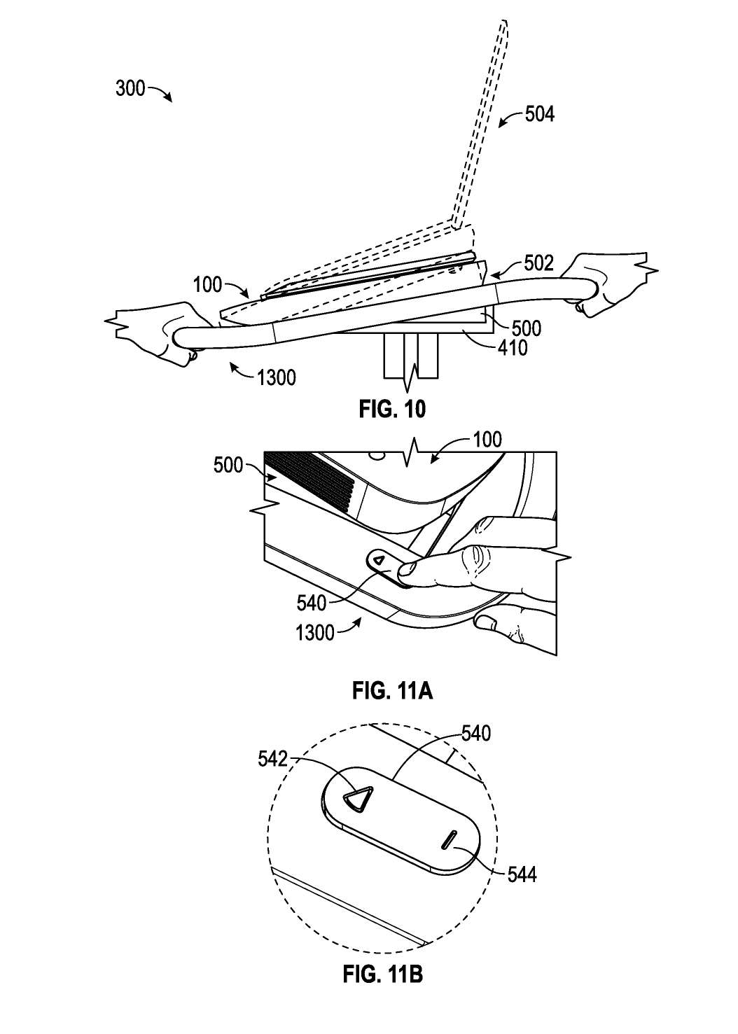

Referring now to FIG. 10, in one embodiment, support structure 500 is automatically adjusted via control circuit 415 to a first position (e.g., starting position, etc.), shown at rest position 502. Support structure 500 may be adjusted to rest position 502 when portable ultrasound system 300 is powered off and/or portable ultrasound device 100 is decoupled from portable ultrasound cart 400. In one embodiment, rest position 502 is at a 10 degree angle with respect to a horizontal (e.g., the floor, etc.). In an alternative embodiment, the rest position 502 is at 0 degrees. In other embodiments, rest position 502 is defined by the operator (e.g., predefined preferences, etc.). By way of example, support structure 500 may not be able to be adjusted to an angle less than rest position 502. By way of another example, setting the rest position 502 at 10 degrees instead of 0 degrees, the volume of support structure 500 and housing 410 is reduced (e.g., in half, etc.). The reduction in volume saves space, facilitating a smaller, lighter, and more maneuverable portable ultrasound cart 400. From rest position 502, support structure 500 may be tilted to increase the angle up to a second position, shown as maximum tilt position 504. In one embodiment, maximum tilt position 504 is at a 20 degree angle. In other embodiments, maximum tilt position 504 is greater than or less than 20 degrees.

Referring back to FIGS. 4A-4B, vertical support member 420 includes a first member, shown as upper support member 422, and a second support member, shown as lower support member 424. In one embodiment, upper support member 422 is positioned within lower support member 424, providing a telescoping feature for vertical support member 420. The telescoping feature of vertical support member 420 may allow for an operator (e.g., an ultrasound technician, a doctor, etc.) to variably adjust the height of portable ultrasound cart 400. The variable height may facilitate the use of portable ultrasound system 300 for different height operators (e.g., short, tall, etc.) and for operators who prefer a standing position or a sitting position. As shown in FIG. 4B, in some embodiments, vertical support member 420 includes an actuator (e.g., a gas shock, an electric motor, etc.), such as height adjustment actuator 426. Height adjustment actuator 426 may be positioned to facilitate the extension and retraction of upper support member 422 with respect to lower support member 424. In some embodiments, the height adjustment actuator 426 adjusts the height of portable ultrasound cart 400 responsive to an input from an operator (e.g., via a button command, a lever actuation, a command on portable ultrasound device 100, etc.). In other embodiments, control circuit 415 automatically provides commands to height adjustment actuator 426 based on predefined operator preferences (as described above) or the state of the portable ultrasound system 300 (e.g., ON, OFF, etc.). The operator preferences may be stored in memory 419 and may be provided to height adjustment actuator 426 responsive to an indication (e.g., identification, etc.) of who is operating portable ultrasound system 300. By way of example, control circuit 415 may automatically adjust the height to an operators preferred setting when portable ultrasound system 300 is powered ON (e.g., based on IP address, serial number, and/or user profile of portable ultrasounds device 100, etc.). By way of another example, control circuit 415 may provide a command to height adjustment actuator 426 to reduce the height of portable ultrasound cart 400 to the minimum height when portable ultrasound system 300 is powered OFF. By reducing the height, portable ultrasound system 300 may be more easily stored (e.g., due to smaller dimensions, etc.), or have increased maneuverability and stability when being moved from one place to another.

Referring still to FIG. 4A, base member 430 includes a plurality of legs 432. Each of the plurality of legs 432 include a caster, shown as casters 434. Base member 430 may include any number of legs 432 and corresponding casters 434 (e.g., 3, 4, 5, 6, etc.). Casters 434 facilitate the movement (e.g., relocation, etc.) of portable ultrasound system 300. Legs 432 of base member 430 provide stability for portable ultrasound system 300, both when stationary and when being moved. Base member 430 may also facilitate the rotation of portable ultrasound system 300 by an operator. Portable ultrasound system 300 may further include a locking mechanism configured to lock portable ultrasound cart 400 in place when in a stationary configuration and be released when in a moving configuration.

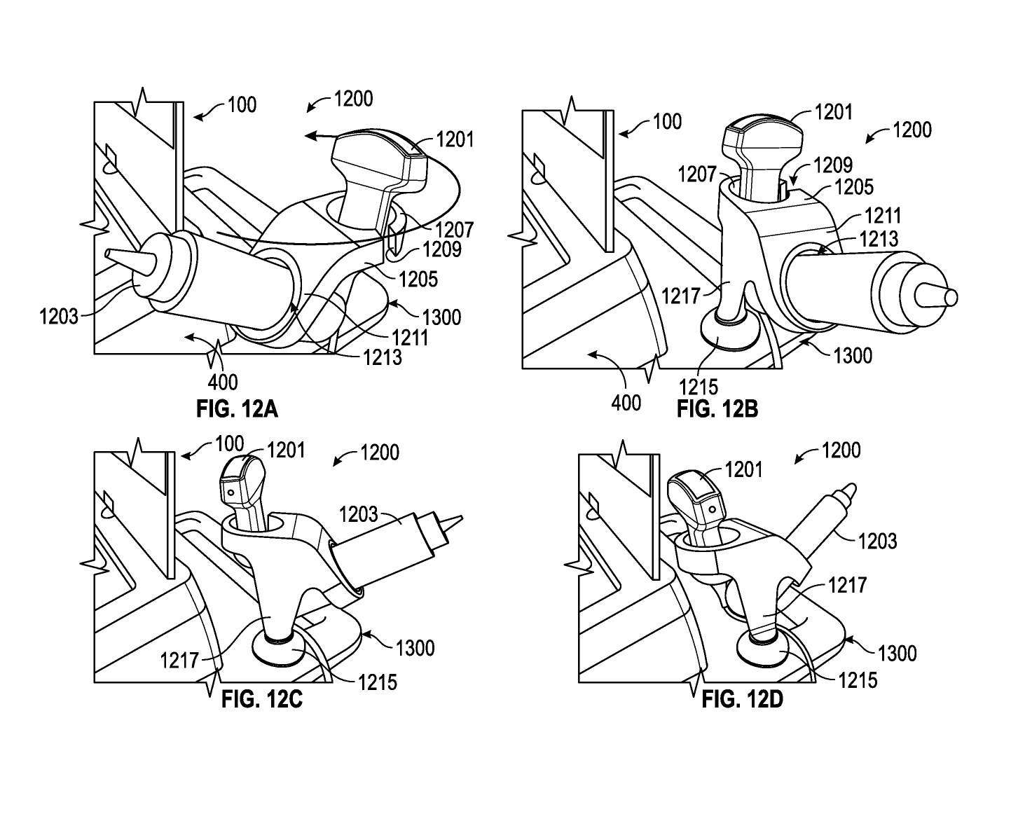

Referring now to FIGS. 4A-4B and 12A-12D, portable ultrasound cart 400 includes transducer housing 1200 positioned along handrail 1300. In one embodiment, portable ultrasound cart 400 includes one transducer housing 1200. In other embodiments, portable ultrasound cart 400 includes a plurality of transducer housings 1200 (e.g., two, three, four, etc.). Portable ultrasound cart 400 may include one transducer housing 1200 on each side to accommodate left handed and right handed users. As shown in FIGS. 12A-12D, transducer housing 1200 includes a first portion, shown as upper portion 1205, and a second portion, shown as lower portion 1211. Upper portion 1205 defines a plane substantially parallel to handrail 1300 (e.g., a horizontal plane, etc.). Lower portion 1211 defines a plane at an angle (e.g., an obtuse angle, etc.) to upper portion 1205. In other embodiments, lower portion 1211 and upper portion 1205 define a single plane (e.g., a flat surface, etc.).

As shown in FIGS. 12A-12D, upper portion 1205 defines a first opening 1207 including a slot 1209. First opening 1207 is structured to hold items or tools that an operator may use while operating portable ultrasound system 300. First opening 1207 may be configured to hold a transducer, shown as transducer 1201. Transducer 1201 may be structured as any type of ultrasound transducer with any suitable type of connection mechanism (e.g., socket type connector, a pin type connector, etc.). In some embodiments, first opening 1207 extends through the entire upper portion 1205 to facilitate a wire/cable extending from transducer 1201 to portable ultrasound cart 400 or portable ultrasound device 100 (e.g., transducer/probe interface 185, etc.). Slot 1209 is configured to allow the wire/cable of transducer 1201 to be removed from first opening 1207 during use by an operator. In other embodiments, transducer 1201 may be wireless such that slot 1209 may be omitted.

According to an example embodiment, transducer 1201 is configured to image a patient by producing an emitting ultrasonic waves. The transducer 1201 measures returning echoes of these waves to provide data regarding the patient. The data may be analyzed and assembled into an image of the patient using a portable ultrasound device 100. In one embodiment, the data is transferred via a wired communication protocol such that transducer 1201 is directly coupled to one of portable ultrasound device 100 and portable ultrasound cart 400. By way of example, a cable extending from transducer 1201 may include an adapter compatible with transducer/probe interface 185 of portable ultrasound device 100. Transducer/probe interface 185 enables transducer 1201 to interface with ultrasound module 191. Ultrasound module 191 may therefore receive the data from transducer 1201 to perform functions related to ultrasound imaging to facilitate the display of the imaged area on portable ultrasound device 100. By way of another example, a cable extending from transducer 1201 may include an adapter compatible with a transducer/probe interface of portable ultrasound cart 400. The transducer/probe interface of portable ultrasound cart 400 enables transducer 1201 to interface with portable ultrasound system interface 450. Portable ultrasound system interface 450 may then communicate the data to ultrasound module 191. In another embodiment, the data is transferred via a wireless communication protocol such that transducer 1201 wirelessly transmits the data to at least one of portable ultrasound device 100 and portable ultrasound cart 400.

Referring still to FIGS. 12A-12D, lower portion 1211 defines a second opening 1213. Second opening 1213 is structured to hold items or tools that an operator may use while operating portable ultrasound system 300. In an medical ultrasound embodiment, second opening 1213 may be configured to hold an examination gel, shown as gel 1203, and/or another medical tool or item. Gel 1203 may be applied to a patient during an ultrasound examination. In other embodiments, transducer housing 1200 may include more or less than two openings and have any structure or shape. In some embodiments, portable ultrasound cart 400 includes a plurality of transducer housings 1200. Each transducer housing 1200 may have a different structure and function (e.g., one may be structured to hold transducer 1201, another may be structured to hold gel 1203, etc.).

As shown in FIGS. 12B-12D, transducer housing 1200 includes an extension portion 1217. Extension portion 1217 extends between both upper portion 1205 and lower portion 1211, and interfaces with base portion 1215. The interface between extension portion 1217 and base portion 1215 couples transducer housing 1200 to portable ultrasound cart 400. Base portion 1215 and extension portion 1217 facilitate the selective repositioning (e.g., rotation, etc.) of transducer housing 1200. Transducer housing 1200 is configured to rotate to effectively reduce the width and improve mobility of portable ultrasound cart 400 (e.g., when moving portable ultrasound cart 400 through narrow passages and doorways, etc.), as well as allow an operator ease of access to the various tools/items (e.g., transducer 1201, gel 1203, etc.) when he or she may be using portable ultrasound system 300. As shown in FIGS. 12A-12D, transducer housing 1200 may be selectively repositioned through a range of motion, from a forward facing position (e.g., a clinical use position, ON position, etc.) (see FIG. 12A) to a rear facing position (e.g., a stored position, OFF position, etc.) (see FIG. 12D). In one embodiment, the selective repositioning of transducer housing 1200 is a manual operation. For example, an operator may provide a torque/force to rotate transducer housing 1200 to a position of his/her liking. In another embodiment, the selective repositioning of transducer housing 1200 is a motorized operation. The motorized operation may replace the manual operation or may supplement the manual operation (e.g., transducer housing 1200 may be positioned either manually or through use of an actuator or motor configured to also allow for manual input).

Referring back to FIG. 4B, in some embodiments, portable ultrasound cart 400 includes a transducer actuator, shown as transducer housing rotation actuator 1230. Transducer housing rotation actuator 1230 is positioned to facilitate the rotation of transducer housing 1200. Transducer housing rotation actuator 1230 may be structured as any suitable actuator (e.g., an electric motor, a mechanical spring mechanism, a gas spring mechanism, etc.). In some embodiments, transducer housing rotation actuator 1230 adjusts the position of transducer housing 1200 responsive to an input from an operator (e.g., a button command, a lever actuation, a command on portable ultrasound device 100, etc.). In another embodiment, control circuit 415 automatically provides commands to transducer housing rotation actuator 1230 to reposition transducer housing 1200 based the state of portable ultrasound system 300 (e.g., ON, OFF, etc.). For example, when portable ultrasound system 300 is turned OFF, control circuit 415 may provide a command to transducer housing rotation actuator 1230 to position (e.g., rotate, etc.) transducer housing 1200 into the rear facing position. Control circuit 415 may provide a command to transducer housing rotation actuator 1230 to position (e.g., rotate, etc.) transducer housing 1200 into the front facing position when portable ultrasound system 300 is turned ON. In further embodiments, portable ultrasound cart 400 includes a position sensor, shown as transducer housing position sensor 1220. Transducer housing position sensor 1220 may be configured to monitor the position of transducer housing 1200 such that control circuit 415 knows how much to actuate transducer housing rotation actuator 1230 when moving between the various positions. For example, an operator may manually rotate transducer housing 1220. Transducer housing position sensor 1220 may determine the current orientation of transducer housing and communicate this information to control circuit 415. Therefore, when a command is provided to actuate transducer housing rotation actuator 1230, control circuit 415 knows how much to rotate transducer housing 1200 from the current location to the desired location (e.g., front facing position, rear facing position, etc.).

In an alternative embodiment, transducer housing position sensor 1220 may further be configured to track the location of an operator such that control circuit 415 automatically adjusts the position of transducer housing 1200 based on the location of the operator. For example, an operator may move relative to portable ultrasound system 300. Therefore, by automatically actuating transducer housing rotation actuator 1230 responsive to the movements of the operator, the tools (e.g., gel 1203, transducer 1201, etc.) stored within transducer housing 1200 may be more readily accessible (e.g., within the reach of the operator, etc.) by the operator.

In one embodiment, transducer housing 1200 is further configured to partially extend (e.g., away from portable ultrasound cart 400) to make an ultrasound tool more accessible to a user (e.g., transducer 1201, gel 1203, etc.). In some embodiments, transducer housing 1200 includes a telescoping segment aligned horizontally relative to portable ultrasound cart 400. In one embodiment, the telescoping segment connects extension portion 1217 of transducer housing 1200 to base portion 1215 or directly to portable ultrasound cart 400. The telescoping segment may pivot relative to base portion 1215 (e.g., is secured to base portion 1215 by a pin or a transducer housing rotation actuator 1230) or pivot relative to the remainder of portable ultrasound cart 400 (e.g., the telescoping segment connects to portable ultrasound cart 400 via a pin or a transducer housing rotation actuator 1230). In alternative embodiments, the telescoping segment is included as a portion of upper portion 1205 or lower portion 211. This allows for the corresponding portion of transducer housing 1200 to move horizontally relative to the remainder of transducer housing 1200. In some embodiments, the telescoping segment includes concentric sections which fit (e.g., with a running fit, interference fit, or other type of fit) within one another such that the sections slide relative to one another. A lip or other feature may be included to prevent one concentric section from pulling out of another section. In some embodiments, the telescoping segment is actuated by a manual force from a user. In alternative embodiments, the telescoping segment is actuated by an actuator configured to control the horizontal movement (e.g., extension and retraction) of the telescoping segment. For example, the actuator may be a linear actuator, drive gear and worm gear combination, and/or other actuator configured to control or provide linear motion. In still further embodiments, the telescoping segment is configured to be adjustable (e.g., extended or retracted) based on either manual force from a user or using an actuator. The actuator may be configured such that the actuator is not damaged if the telescoping segment is manually adjusted by a user. The horizontal position of transducer housing 1200 and/or a portion thereof may be adjusted in the same or similar fashion as the rotational position of transducer housing 1200 described herein. For example, the horizontal position (e.g., extension of the telescoping segment) may be automatically adjusted based on user preferences or settings upon determining the identity of a user, the horizontal position may be adjusted to a rest position when portable ultrasound system 300 is powered off and/or portable ultrasound device 100 is decoupled from portable ultrasound cart 400, the horizontal position may be adjusted when portable ultrasound device 100 is coupled to portable ultrasound cart 400 or turned ON based on a user identity, and/or the horizontal position may otherwise be adjusted.

Referring again to FIG. 4B, portable ultrasound cart 400 may include power source 440. Power source 440 is configured to provide power to the various components of portable ultrasound cart 400 (e.g., control circuit 415, support structure tilt actuator 530, transducer housing rotation actuator 1230, etc.) to perform the various function described herein. In one embodiment, power source 440 is an internal power source stored within housing 410 of portable ultrasound cart 400. For example, power source 440 may be structured as a rechargeable or non-rechargeable battery. In another embodiment, power source 440 is an external power source. For example, portable ultrasound cart 400 may include a retractable or non-retractable power cable with any suitable plug adapter (e.g., a type A-type O adapter, etc.) configured to couple to an external power source (e.g., a generator, a wall outlet, mains power, etc.). In another embodiment, one of portable ultrasound device 100 and portable ultrasound cart 400 draws power from the other. By way of example, power source 440 of portable ultrasound cart 400 may charge portable ultrasound device 100 when coupled together (e.g., via interfacing components on support structure 500 and portable ultrasound device 100, via a charging cord/cable such as power adapter 178 extending from portable ultrasound cart 400 to power input 176 of portable ultrasound device 100, etc.). By way of another example, portable ultrasound device 100 may provide power to portable ultrasound cart 400 to operate the various components of the portable ultrasound cart 400.

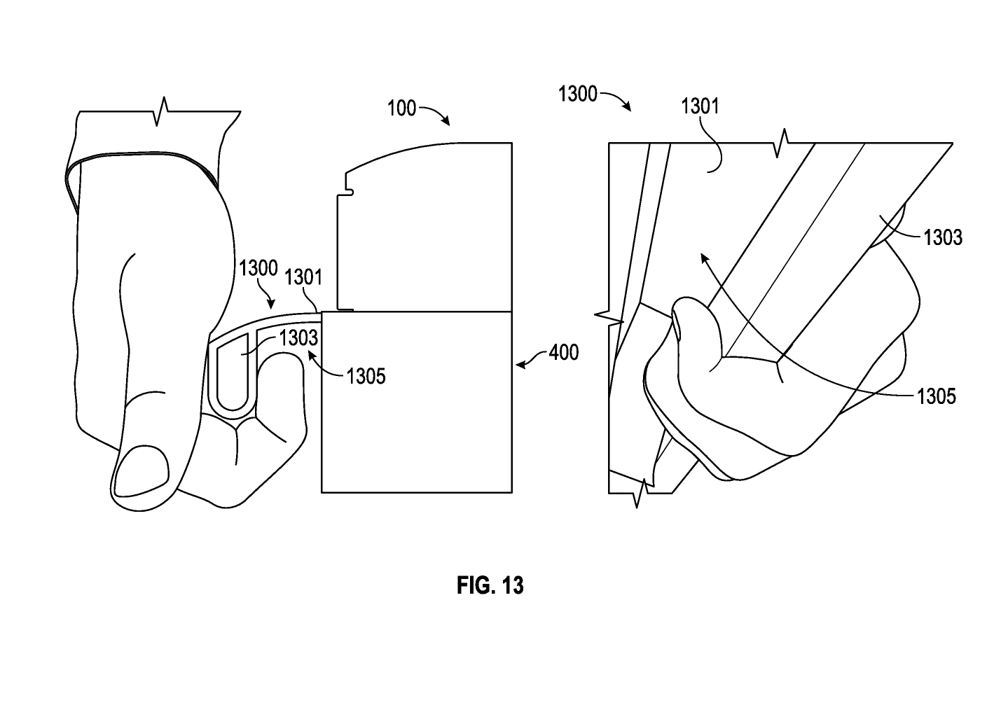

Referring now to FIGS. 4A and 13, portable ultrasound cart 400 includes handrail 1300. In one embodiment, handrail 1300 extends around the periphery of portable ultrasound cart 400. In some embodiments, handrail 1300 is a single, continuous structure that extends around the entire periphery of portable ultrasound cart 400. In other embodiments, handrail 1300 includes a front handle, a rear handle, a right handrail, and a left handrail, where each may have a different shape. In either case, handrail 1300 may provide 360 degree handhold access for ease of maneuverability of portable ultrasound cart 400. As shown in FIG. 13, handrail 1300 includes upper portion 1301 and side portion 1303. Upper portion 1301 and side portion 1303 are structured to provide an undercut form that defines a recess, shown as recess 1305. The structure of handrail 1300 provides a comfortable fit for a hand of an operator of portable ultrasound system 300 such that when the operator grips handrail 1300, his/her fingers extend into recess 1305. Recess 1305 may be substantially rectangular with a concave top section. Recess 1305 may have a width (e.g., defined by the width of upper portion 1301 and the width of side portion 1303) which is configured to accommodate a finger of a hand. For example, recess 1305 may have a width of one and a half fingers, two fingers, and/or other widths to accommodate a user's fingers. Upper portion 1301 may slope down and away from portable ultrasound cart 400 to provide for a comfortable grip and reduce corners or sharp edges. The transition between upper portion 1301 and side portion 1303 may be sharp, rounded, radiused, chamfered, or have other configurations. Side portion 1303 may have a lower surface with is rounded. The width of side portion 1303 may be configured to provide a comfortable grip for a user. For example, the width of side portion 1303 may be one, two, or three finger widths or may have a different width.

Referring to FIGS. 4A and 13, handrail 1300 may be coupled to the sides of housing 410 of portable ultrasound cart 400. Upper portion (e.g., surface) 1301, side portion 1303, and/or recess 1305 provide for a surface/structure which allows a user to push, grab, pull, and/or otherwise manipulate portable ultrasound cart 400 from the side. In some embodiments, there is substantially no gap between upper portion 1301 and the sides of portable ultrasound cart 400 (e.g., housing 410) and there is a gap between upper portion 1301 and the front and/or rear of portable ultrasound cart 400. This may allow a user to grasp upper portion 1301 and side portion 1303 with a user's hand extending over and/or around upper portion 1301 without being within recess 1305. Advantageously, this may allow for a user to quickly and/or easily push or pull ultrasound cart 400 without placing their fingers in recess 1305. In other embodiments, there is substantially no gap between upper portion 1301 and all sides of portable ultrasound cart 400 (e.g., housing 410).

According to an example embodiment, portable ultrasound cart 400 provides a substantially ergonomic configurability for an operator. As described above, various components of portable ultrasound cart 400 provide height adjustment of portable ultrasound cart 400 (e.g., via height adjustment actuator 426, etc.), tilt adjustment of support structure 500 (e.g., via support structure tilt actuator 530, etc.), and orientation adjustment of transducer housing 1200 (e.g., via transducer housing rotation actuator 1230, etc.). In one embodiment, an operator is able to configure portable ultrasound cart 400 via various buttons, levers, and/or commands from portable ultrasound device 100. In another embodiment, operator predefined preferences are stored within memory 419 of portable ultrasound cart 400 or memory 165 of portable ultrasound device 100. For example, when portable ultrasound system 300 is turned ON or an new operator begins to use portable ultrasound system 300 (e.g., a portable ultrasound device 100 is coupled to portable ultrasound cart 400, etc.), control circuit 415 may automatically reconfigure portable ultrasound cart 400 into a first mode, such as an operation mode. In the first mode, portable ultrasound cart 400 is reconfigured based on the preferred settings of the operator using portable ultrasound system 300. In other embodiments, portable ultrasound system 300 is reconfigured into a second mode (e.g., such as a storage mode, park mode, OFF mode, or relocation mode, etc.), when portable ultrasound system 300 is turned OFF. In the second mode, portable ultrasound cart 400 is reconfigured to reduce the size and increase the maneuverability/stability of portable ultrasound cart 400 (e.g., transducer housing 1200 is repositioned to the rear facing position, the height of vertical support member 420 is minimized, the support structure 500 is returned to rest position 502, etc.).

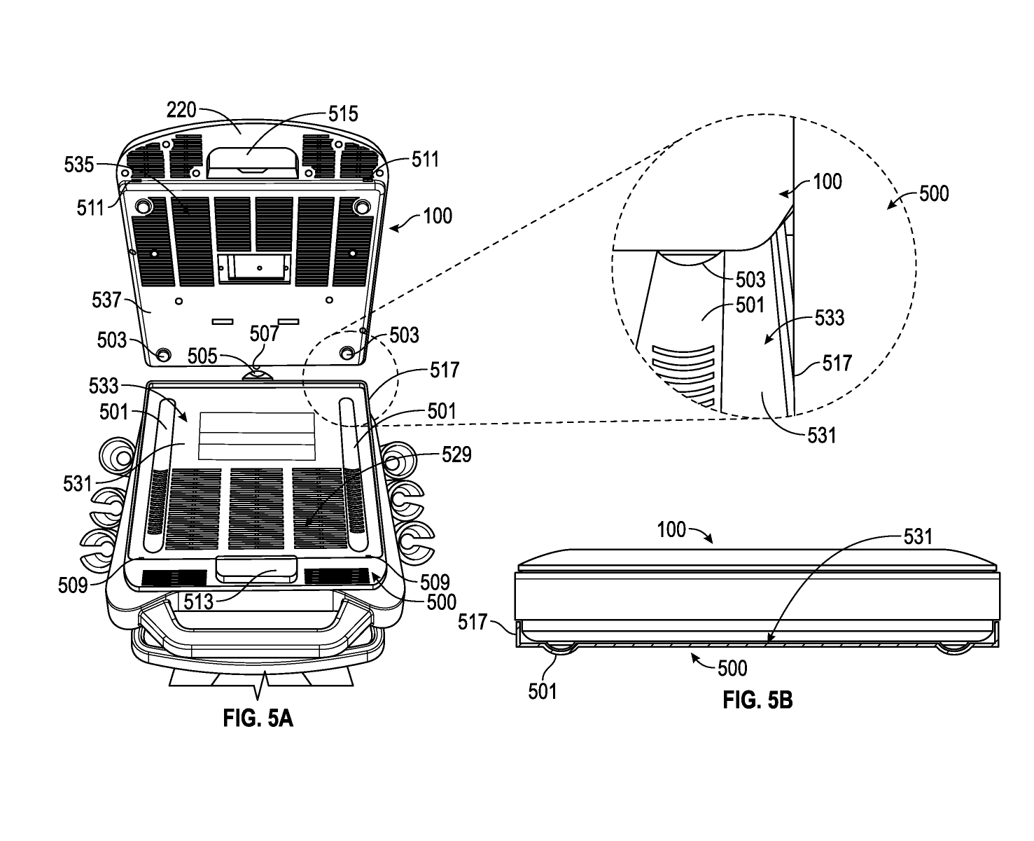

Referring now to FIGS. 5A-7B, portable ultrasound cart 400 includes features configured to releasably secure portable ultrasound device 100 to portable ultrasound cart 400. As shown in FIGS. 5A-5B, support structure 500 includes a planar surface, shown as planar surface 531. Planar surface 531 provides a mounting surface for bottom surface 537 of portable ultrasound device 100 to rest upon when secured (e.g., coupled, etc.) to portable ultrasound cart 400. In some embodiments, planar surface 531 defines grooves 501 structured to receive feet 503 of portable ultrasound device 100. Advantageously, grooves 501 may be configured to receive feet 503 such that portable ultrasound device 100 may be guided into position using a single hand. Grooves 501 receive feet 503 and align portable ultrasound device 100 with portable ultrasound cart 400 such that connectors 505 and 509 of portable ultrasound cart 400 are aligned with connectors 507 and 511 of portable ultrasound device 100. Furthermore, portable ultrasound device 100 is aligned with portable ultrasound cart 400 such that bottom layer 805 of portable ultrasound device 100 is aligned with walls 517 of portable ultrasound cart 400 and bottom layer 805 may be received into the space defined by walls 517 and planar surface 531. As shown in FIGS. 5A-5B, support structure 500 includes walls 517 positioned around the periphery of support structure 500. Walls 517 and planar surface 531 define recess 533 configured to receive portable ultrasound device 100 such that bottom layer 805 is contained within recess 533.

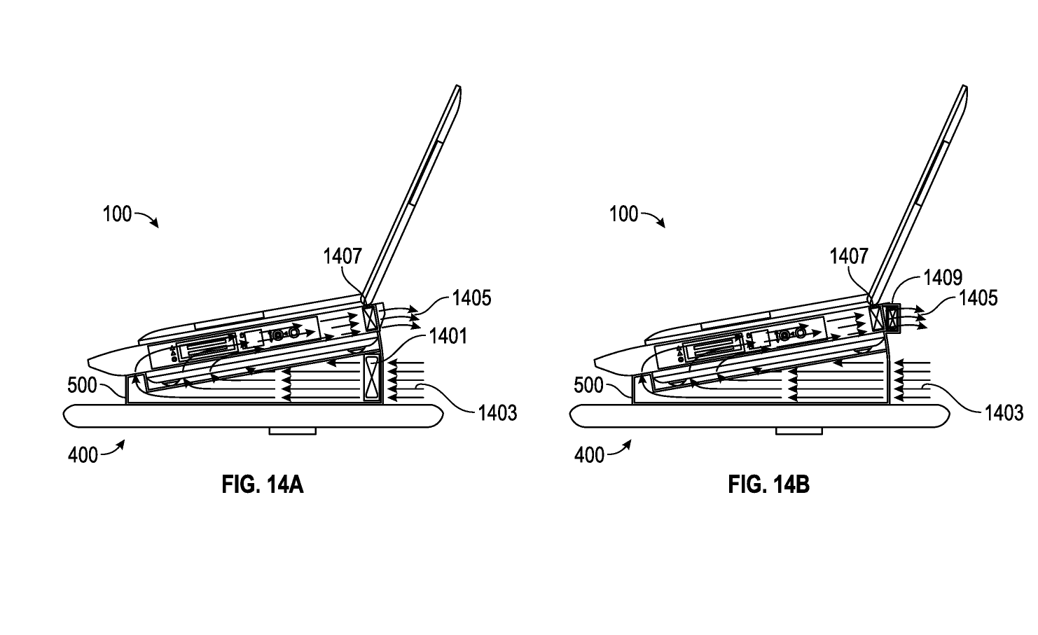

In some embodiments, planar surface 531 defines slots 529 configured to interface with slots 535 defined by bottom surface 537 of portable ultrasound device 100 (e.g., for thermal management, for air flow, etc.). In some embodiments, slots 529 align with slots 535. In alternative embodiments, slots 529 do not align with slots 535 but do provide for airflow through planar surface 531 and into slots 535 of portable ultrasound device 100. These features are discussed in greater detail later herein with reference to FIGS. 14A and 14B.

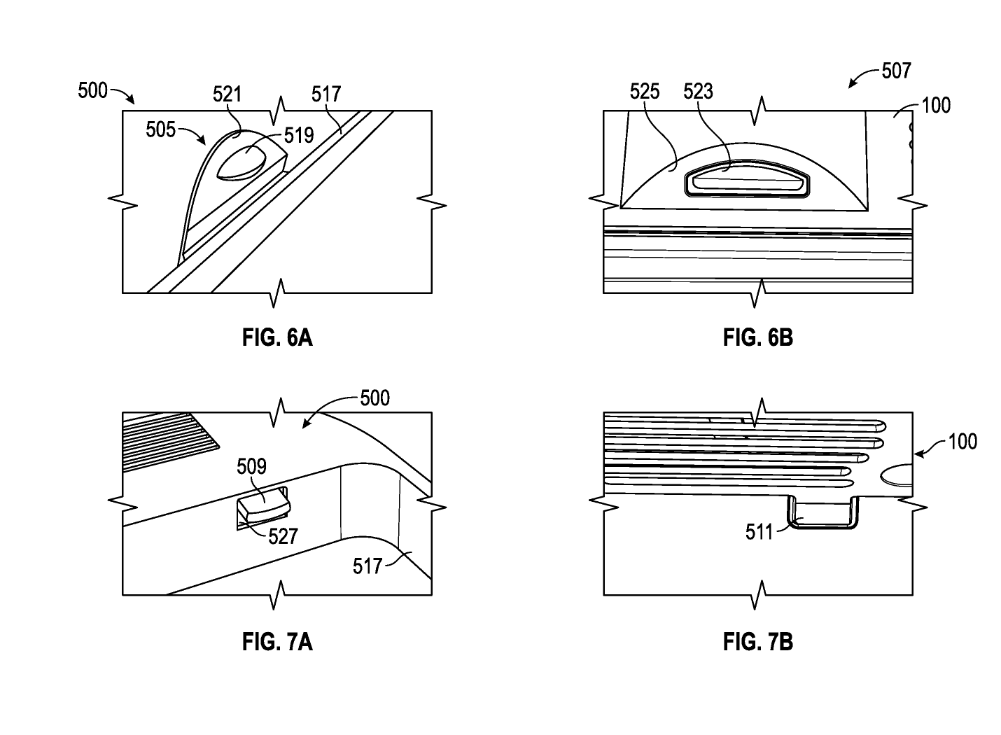

As shown in FIGS. 5A, 6A, and 7A, support structure 500 includes a first male engagement feature, shown as rear male engagement feature 505, and two second male engagement features, shown as front male engagement features 509. In some embodiments, support structure 500 includes a different number of rear male engagement features 505 and/or front male engagement features 509. As shown in FIG. 5A, rear male engagement feature 505 is positioned at the rear of support structure 500 and front male engagement features 509 are positioned at the front of support structure 500. As shown in FIG. 6A, rear male engagement feature 505 includes base 521 with tab 519 extending from base 521. In one embodiment, tab 519 is fixed. In an alternative embodiment, tab 519 is retractable (e.g., spring-loaded, controlled by an actuator, etc.). As shown in FIG. 7A, front male engagement features 509 extend from openings 527. In one embodiment, front male engagement features 509 are spring-loaded to facilitate retraction and extension. As shown in FIG. 5A, support structure 500 further includes release lever 513. In one embodiment, release lever 513 is linked to front male engagement features 509 such that release lever 513 is configured to actuate front male engagement features 509 to at least one of extend and retract.

As shown in FIGS. 5A, 6B, and 7B, portable ultrasound device 100 includes a first female engagement feature, shown as rear female engagement features 507, and two second female engagement features, shown as front female engagement features 511. In some embodiments, portable ultrasound device 100 includes a different number of rear female engagement features 507 and/or front female engagement features 511 (e.g., to correspond with the number of rear male engagement features 505 and front male engagement features 509 of support structure 500, etc.). As shown in FIG. 5A, rear female engagement features 507 is positioned at the rear of portable ultrasound device 100 and front female engagement features 511 are positioned at the front of portable ultrasound device 100. As shown in FIG. 6B, rear female engagement features 507 includes base 525 that defines an aperture 523. As shown in FIG. 7B, front female engagement features 511 are apertures defined by portable ultrasound device 100.

According to the example embodiment, rear male engagement feature 505 of support structure 500 is positioned to interface with rear female engagement feature 507 of portable ultrasound device 100. Front male engagement features 509 of support structure 500 are positioned to interface with front female engagement features 511 of portable ultrasound device 100. In an alternate embodiment, the engagement features of portable ultrasound device 100 are male engagement features and the engagement features of support structure 500 are female engagement features. In yet another embodiment, both portable ultrasound device 100 and support structure 500 include at least one of a male engagement feature and a female engagement feature. In alternative embodiments, portable ultrasound device 100 couples to support structure 500 via another type of engagement. By way of example, the engagement may include a magnetic type of coupling. By way of another example, the engagement may include a hook and loop type of coupling. By way of yet another example, the engagement may include another type of coupling or no coupling (e.g., portable ultrasound device 100 rests within recess 533 and retained by walls 517, etc.).

The aforementioned structure of support structure 500 and portable ultrasound device 100 facilitate a substantially easy coupling of portable ultrasound device 100 to portable ultrasound cart 400 such that the coupling may be done one handed. For example, support structure 500 can be configured to enable a user to couple (e.g., engage, install, secure etc.) portable ultrasound device 100 to portable ultrasound cart 400 with one hand. Support structure 500 can include one or more engagement features that automatically engage portable ultrasound device 100 in response to contact with portable ultrasound device 100 (or features thereof). For example, front male engagement features 509 of support structure 500 can automatically engage front female engagement features 511 of portable ultrasound device 100. Portable ultrasound device 100 can be automatically secured to portable ultrasound cart 400 in response to interfacing of portable ultrasound device 100 and portable ultrasound cart 400 (or in response to interfacing of components thereof, such as described herein), which can enable a user to couple portable ultrasound device 100 to portable ultrasound cart 400 with one hand (e.g., without requiring a second hand that is not holding portable ultrasound device 100 to engage an actuator of portable ultrasound cart 400). In some embodiments, portable ultrasound cart 400 is configured to automatically secure (e.g., engage, be coupled to, etc.) portable ultrasound device 100 in response to manipulation (e.g., sliding, pushing, pressing, etc.) of portable ultrasound device 100 on a surface of portable ultrasound cart 400. According to an example embodiment, handle system 220 of portable ultrasound device 100 defines aperture 515 that facilities the carrying of portable ultrasound device 100 with one hand.

By way of example, securing portable ultrasound device 100 to portable ultrasound cart 400 may be as follows. An operator may lift portable ultrasound device 100 via handle system 220 into recess 533 of support structure 500. Both walls 517 and grooves 501 orient portable ultrasound device 100 onto support structure 500 such that feet 503 interface with grooves 501 and the edges of portable ultrasound device 100 abut walls 517. The operator slides portable ultrasound device 100 along grooves 501 until base 525 of rear female engagement features 507 of portable ultrasound device 100 interfaces with base 521 of rear male engagement feature 505 of support structure 500 such that aperture 523 receives tab 519. The front end (e.g., the end with handle system 220, etc.) of portable ultrasound device 100 may then be lowered such that front male engagement features 509 of support structure 500 interface with front female engagement features 511 of portable ultrasound device 100. As front female engagement features 511 of portable ultrasound device 100 interface with front male engagement features 509 of support structure 500, front male engagement features 509 retract into openings 527. When bottom surface 537 of portable ultrasound device 100 rests flush with planar surface 531 of support structure 500 (see, e.g., FIG. 5B), front male engagement features 509 extend from openings 527 such that front female engagement features 511 of portable ultrasound device 100 receive front male engagement features 509, thereby securing portable ultrasound device 100 to support structure 500. When coupled, aperture 515 of handle system 220 receives release lever 513 such that release lever 513 is accessible through aperture 515. Release lever 513 may be used to decouple (e.g., release, detach, disengage, etc.) portable ultrasound device 100 from support structure 500. For example, by pressing on release lever 513, front male engagement features 509 of support structure 500 may disengage from front female engagement features 511, thereby facilitating the removal of portable ultrasound device 100 from recess 533 of support structure 500. A user may reach into aperture 515 and actuate release lever 513 to disengage front male engagement features 509 and lift portable ultrasound device 100 using handle system 220 which defines aperture 515 as the user's hand is already within aperture 515 and in position to grasp handle system 220 through aperture 515 as portable ultrasound device 100 is lifted away from support structure 500. Release lever 513 is co-located with handle system 220 of portable ultrasound device 100 when portable ultrasound device 100 is secured to portable ultrasound cart 400. For example, this provides for the release operation described herein. Advantageously, the configuration (e.g., positioning) of the release lever 513 and other features of portable ultrasound cart 400 described herein (e.g., the positioning of male engagement features 509, grooves 501, rear male engagement feature 505, walls 517, and/or other features) may allow for one handed grasping of handle system 220 of portable ultrasound device 100 and actuation of release lever 513. This allows a user to disengage portable ultrasound device 100 from portable ultrasound cart 400 using a single hand while lifting portable ultrasound device 100 away from portable ultrasound cart 400 using that hand and handle system 220 of portable ultrasound device 100. In some embodiments, release lever 513 is positioned to enable a user to coupled portable ultrasound device 100 to portable ultrasound cart 400 (e.g., release lever 513 does not interfere with coupling of portable ultrasound device 100 to portable ultrasound cart 400; release lever 513 automatically transitions to a position in which front male engagement features 409 can engage front female engagement features 511 to a position in which actuation of release lever 513 causes front male engagement features 409 to disengagement front female engagement features 511, in response to portable ultrasound device 100 being received by portable ultrasound cart 400). For example, as portable ultrasound device 100 is slid into position (e.g., a final position, an operating position) on portable ultrasound cart 400, portable ultrasound device 100 can be automatically latched (e.g., secured, engaged, locked) into place with respect to portable ultrasound cart 400 without any additional actuation. In some embodiments, the grooves 501 are configured to align portable ultrasound device 100 on portable ultrasound cart 400 (or align features thereof) such that portable ultrasound device 100 can be automatically secured to portable ultrasound cart 400 in response to portable ultrasound device 100 interfacing portable ultrasound cart 400 (e.g., without any additional actuation by a user).

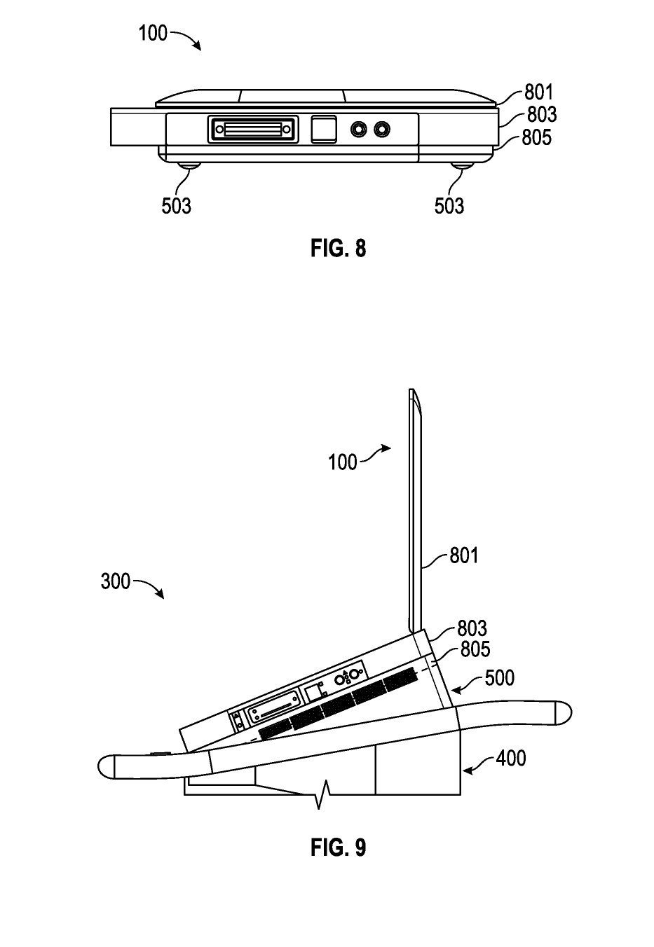

Referring now to FIGS. 8-9, the coupling of portable ultrasound device 100 to support structure 500 provides a substantially integrated appearance. As shown in FIG. 8, portable ultrasound device 100 includes top layer 801, middle layer 803, and bottom layer 805. Top layer 801 includes display housing 140 and main screen 130. Middle layer 803 includes the user interface (e.g., touchscreen 110, touchscreen 120, etc.), handle system 220, locking lever system 210, and the internal hardware components (see, e.g., FIG. 3). Bottom layer 805 includes feet 503 and additional internal hardware components. As shown in FIG. 9, bottom layer 805 is hidden when portable ultrasound device 100 is coupled to support structure 500 of portable ultrasound cart 400. Hiding bottom layer 805 makes the portable ultrasound device 100 appear smaller and highly integrated with the portable ultrasound cart 400 such that the two appear to be joined as a single entity.

Referring now to FIGS. 5A and 14A-14B, portable ultrasound cart 400 may include a thermal management system. The thermal management system is configured enhance the thermal management capability of the portable ultrasound device 100. In one embodiment, the thermal management system is coupled to support structure 500. As described above in regard to FIG. 5A, planar surface 531 of support structure 500 defines slots 529 positioned to interface with slots 535 defined by bottom surface 537 of portable ultrasound device 100. The interfacing slots 529 and 535 provide passage ways for air to flow to facilitate the removal of thermal energy (e.g., heat, etc.) from the internal components of at least one of portable ultrasound device 100 and portable ultrasound cart 400. As shown in FIGS. 14A-14B, portable ultrasound device 100 includes an internal fan, shown as fan 1407, configured to pull heat from the internal components positioned within portable ultrasound device 100. According to the example embodiment shown in FIG. 14A, support structure 500 includes a first supplemental fan, shown as fan 1401, configured to push ambient air 1403 (e.g., cool air, etc.) into portable ultrasound device 100. Fan 1401 is positioned upstream of the fan 1407 to increase airflow through the passageway defined between the interfacing surfaces of portable ultrasound device 100 (e.g., bottom surface 537, etc.) and portable ultrasound cart 400 (e.g., planar surface 531 of support structure 500, etc.) when coupled together. Portable ultrasound device 100 receives the cool air via the interface between slots 529 and 535, cooling the internal components. Fan 1407 then pulls heated air 1405 from portable ultrasound device 100. According to the example embodiment shown in FIG. 14B, portable ultrasound device 100 receives ambient air 1403 via the interface between slots 529 and 535, cooling the internal components. As shown in FIG. 14B, support structure 500 includes a second supplemental fan, shown as fan 1409, configured to aid (e.g., supplement, etc.) fan 1407 in pulling heated air 1405 out of portable ultrasound device 100. Fan 1409 is positioned downstream of fan 1407 to increase airflow through the passageway defined between the interfacing surfaces of portable ultrasound device 100 (e.g., bottom surface 537, etc.) and portable ultrasound cart 400 (e.g., planar surface 531 of support structure 500, etc.) when coupled together. In an alternative embodiment, support structure 500 includes both fan 1401 configured to push ambient air 1403 into portable ultrasound device 100 and fan 1409 configured to pull heated air 1405 out of portable ultrasound device 100 to supplement internal fan 1407. In still further embodiments, support structure 500 may include one or more vents located on one or more sides of support structure 500.

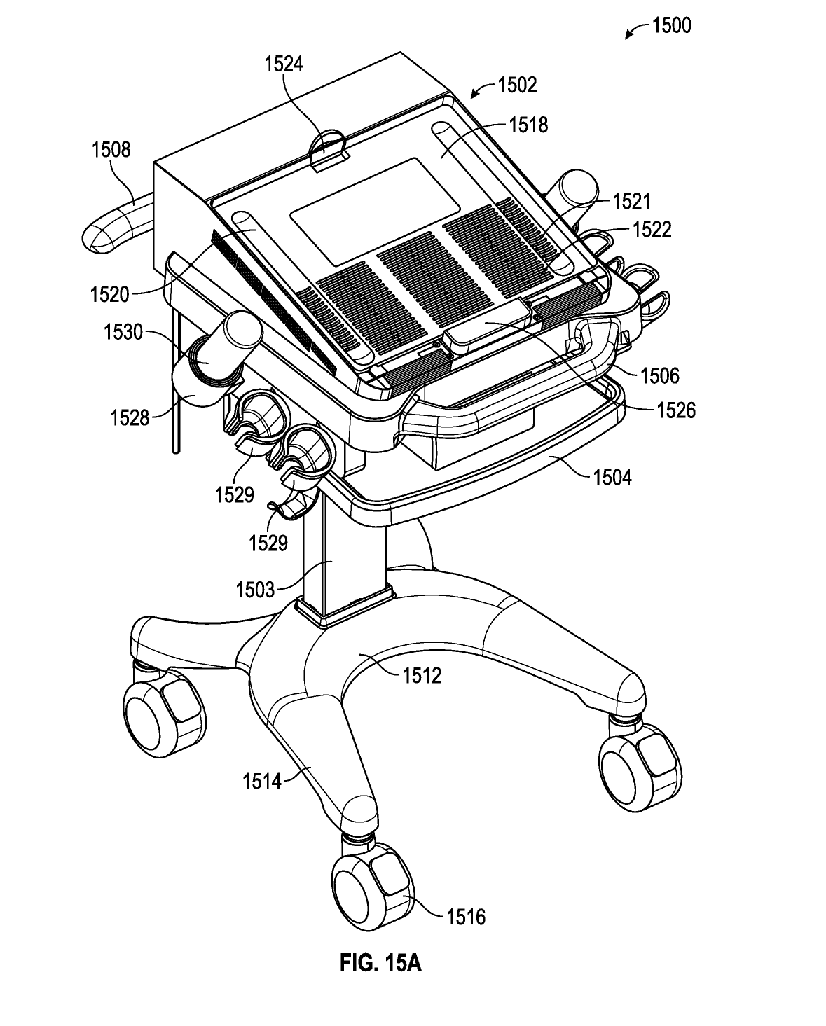

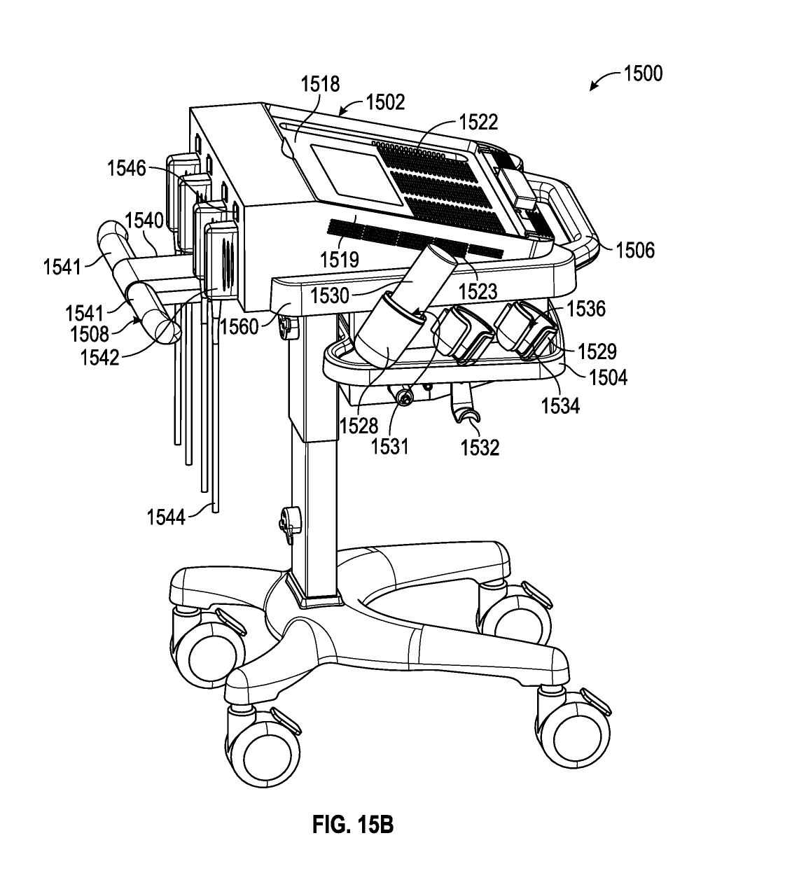

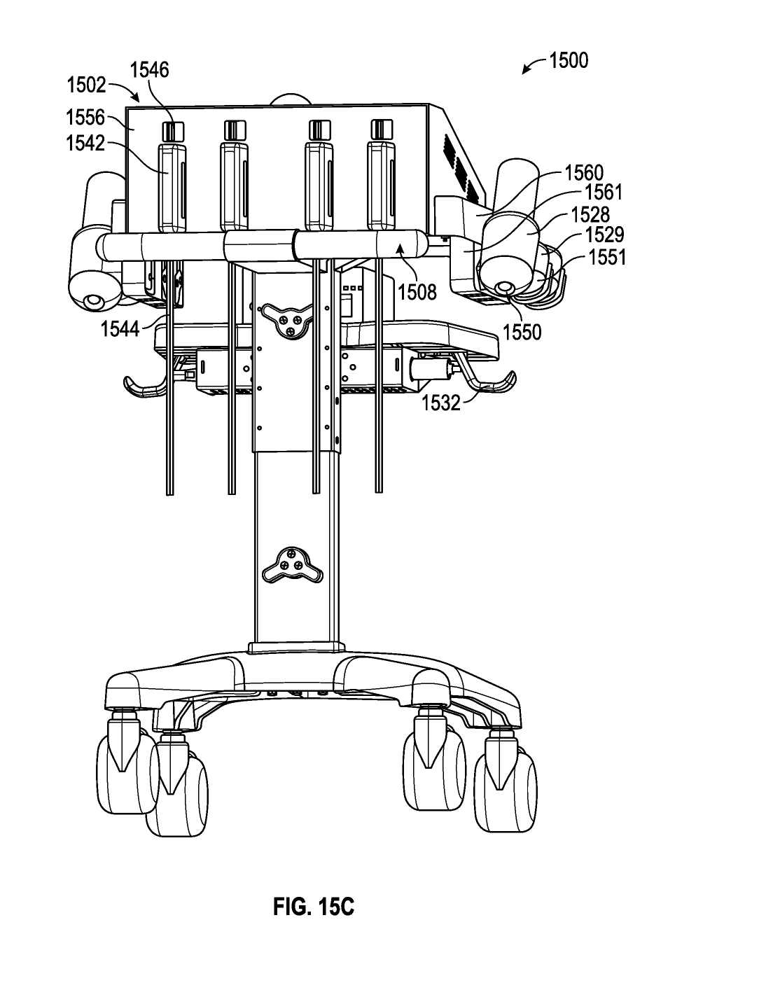

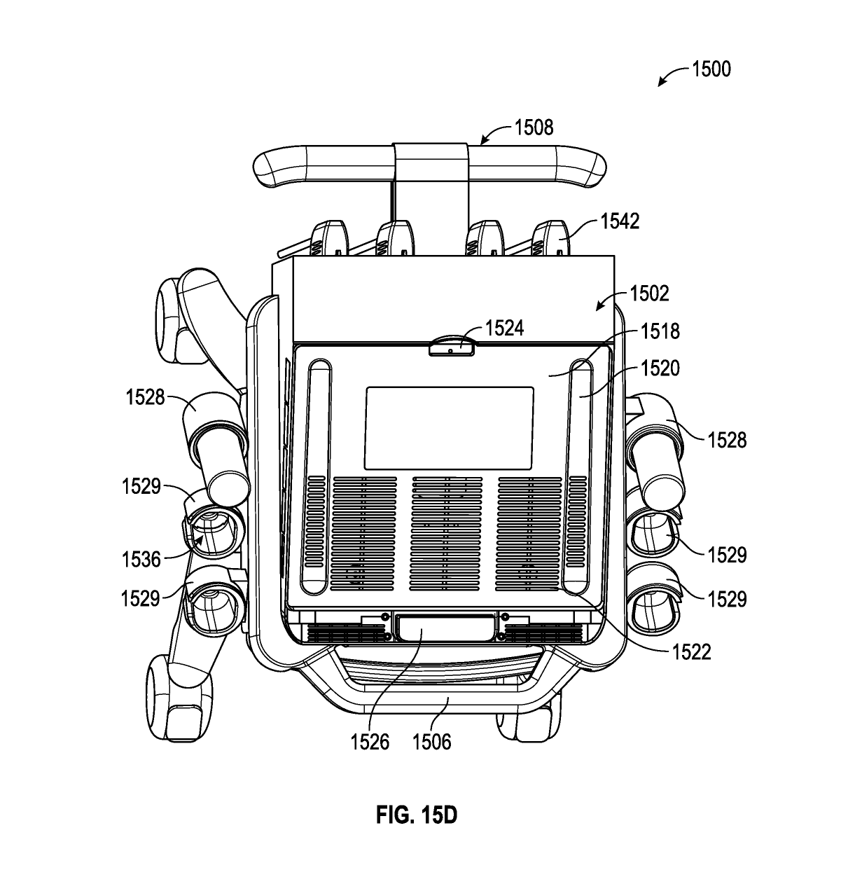

Referring now to FIGS. 15A-15D, portable ultrasound cart 1500 is illustrated. Portable ultrasound cart 1500 may be provided with various structural and functional features that are similar or identical to portable ultrasound cart 400 as illustrated in FIGS. 4A-4B and disclosed herein, including relative dimensions, angles, relationships between components, connections for transmitting and receiving data, etc. Portable ultrasound cart 1500 may be used as part of portable ultrasound system 300, such as in conjunction with a portable ultrasound device such as portable ultrasound device 100.

As shown in FIGS. 15A-15D, portable ultrasound cart 1500 includes support housing (e.g., structure) 1502, vertical support member 1503, and central panel 1504 disposed between support housing 1502 and vertical support member 1503. Vertical support member 1503 extends to base member 1512, from which legs 1514 extend. Legs 1514 terminate in casters 1516.

As shown in FIGS. 15A, 15B, and 15D, support housing 1502 includes planar surface 1518 configured to receive portable ultrasound device 100. Planar surface 1518 is similar to planar surface 531 of support structure 500 as shown in FIGS. 5A-5B. Planar surface 1518 as shown is disposed at an angle relative to a plane defined by central panel 1504 and/or a surface upon which portable ultrasound cart 1500 is disposed. In various embodiments, planar surface 1518 may be disposed at various angles, such as at an angle configured to optimally receive portable ultrasound device 100. Planar surface 1518 includes grooves 1520, with slots 1521 formed in grooves 1520 and slots 1522 formed in planar surface 1518. In various embodiments, the positioning of slots 1521, 1522 may be configured for optimally interacting with heat exhausted by portable ultrasound device 100, such as in a manner similar to the thermal management system illustrated in FIGS. 14A-14B and disclosed herein. Support housing 1502 also includes female engagement member 1524 and male engagement member 1526 for receiving and/or coupling with portable ultrasound device 100. Support housing 1502 may also include handrail 1506 extending from support housing 1502. Central panel 1504 may include hooks 1532 for holding devices such as a transducer.

As shown in FIGS. 15A-15D, portable ultrasound cart 1500 may include transducer housings 1528, 1529 disposed on sides of support housing 1502. Transducer housings 1528, 1529 may receive various objects (e.g. transducers, gels, etc.). Transducer housing 1528, 1529 may be disposed on a “left” and “right” side of support housing 1502 (e.g., relative to a point of view facing handrail 1506). In various embodiments, transducer housings 1528, 1529 may be disposed in various locations, numbers, and orders, such as for facilitating “left-handed” or “right-handed” operation of portable ultrasound cart 1500 (e.g., left-handed operation may place one of transducer housings 1528, 1529 proximate to handrail 1506 on the left side of support housing 1502, and similarly right-handed operation may place one of transducer housings 1528, 1529 proximate to handrail 1506 on the right side of support housing 1502, depending on a user's preferences for which of transducers, gels, or other items are most easily accessible from the handrail 1506 side of portable ultrasound cart 1500). Transducer housings 1528, 1529 are shown to be attached to side rail 1561. Side rail 1561 extends from outer housing 1560. Outer housing 1560 extends from support housing 1502 and may be continuous with handrail 1506. Transducer housings 1528 define cavities 1531 in which an item such as a transducer or gel 1530 may be received. Transducer housings 1528 also define opening 1550 to allow an object such as a wire/cable extending from a transducer to exit transducer housing 1528. Transducer housings 1529 define cavities 1536 in which an item such as a transducer or a gel may be received. Transducer housings 1529 also define slots 1534 configured to allow a wire/cable to be manipulated, such as for placing a transducer in transducer housing 1529 while allowing wire/cable to extend out of slot 1534; slots 1534 also define an opening 1551 similar to opening 1550 to allow a wire/cable or other similar object to exit transducer housing 1529 in a direction towards base member 1512. As shown in FIGS. 15A-15D, transducer housings 1528, 1529 are tilted at an angle relative to an axis parallel to vertical support member 1503, facilitating manipulation by a user of transducers and other objects to be placed in or removed from transducer housings 1528, 1529.

As shown in FIGS. 15A-15B, support housing 1502 includes sidewall 1519 extending from planar surface 1518 towards vertical support member 1503. Sidewall 1519 may be provided with slots 1523. In some embodiments, slots 1523 are in fluid communication with slots 1521 and/or slots 1522. Accordingly, a fluid pathway may be provided, such as for moving air (e.g., hot air produced by heat exhausted from portable ultrasound device 100) from planar surface 1518 out through slots 1523. The fluid pathway including slots 1521, 1522, and/or 1523 may be configured to function in concert with a thermal management system (e.g., the thermal management system shown in FIGS. 14A-14B and disclosed herein).

As shown in FIGS. 15A-15D, rear (e.g., on an opposite side of support housing 1502 from handrail 1506) handle 1508 may extend from rear wall 1556. Rear handle 1508 may include extension 1540 extending directly from rear wall 1556, and handles 1541 extending from an outer (e.g., distal relative to rear wall 1556) end of extension 1540. Handles 1541 may extend perpendicular to extension 1540 and in a plane parallel to central panel 1504. Handles 1541 may include friction grips, foam grips, or other handle features configured to facilitate manipulation of rear handle 1508 and in turn portable ultrasound cart 1500. Rear wall 1556 may also include rear engagement slots (not shown) allowing objects such as transducers 1542 to be coupled and/or engaged to portable ultrasound cart 1500, such as for operably coupling transducers 1542 to portable ultrasound device 100, portable ultrasound cart 1500, and/or any electronic components provided therein. Transducers 1542 may include wires 1544 allowing for electric/electronic connection to transducers 1542. Wires 1544 may hang free when transducers 1542 are coupled to the rear engagement slots, facilitating manipulation of wires 1544, such as when a user intends to connect transducer 1542 to both portable ultrasound cart 1500 and another remote device. In some embodiments, rear wall 1556 includes switches (e.g., toggles, buttons, etc.) 1546 associated with an a rear engagement slot for engaging transducer 1542. For example, switch 1546 may include a first state configured to allow an electrical/electronic connection between transducer 1542 and portable ultrasound cart 1500 and/or portable ultrasound device 100. Switch 1546 may include a second state configured to interrupt or block an electrical/electronic connection between transducer 1542 and portable ultrasound cart 1500 and/or portable ultrasound device 100. Switches 1546 may also be configured to partially or completely lock transducers 1542 to portable ultrasound cart 1500.

The present disclosure contemplates methods, systems, and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general purpose computer, special purpose computer, or special purpose processing machines to perform a certain function or group of functions.

Although the figures may show a specific order of method steps, the order of the steps may differ from what is depicted. Also two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule based logic and other logic to accomplish the various connection steps, processing steps, comparison steps and decision steps.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A portable ultrasound cart, comprising:
a support structure configured to releasably secure a portable ultrasound device, the support structure including:
a wall positioned around a periphery of the support structure;
a planar surface, wherein the wall and the planar surface define a recess, the recess structured to receive a portion of the portable ultrasound device;
a first male engagement feature positioned to engage with a first female engagement feature of the portable ultrasound device; and
a second male engagement feature positioned to engage with a second female engagement feature of the portable ultrasound device;
a tilt system positioned to adjust a tilt angle of the support structure relative to a remainder of the portable ultrasound cart; and
a release lever, wherein the second male engagement feature of the support structure is retractable, and wherein the release lever is linked to the second male engagement feature such that when the release lever is actuated, the second male engagement feature of the portable ultrasound cart disengages from the second female engagement feature of the portable ultrasound device.

2. The portable ultrasound cart of claim 1, wherein the release lever is configured to fit at least partially within an aperture of a handle system of the portable ultrasound device when the portable ultrasound device is engaged with the portable ultrasound cart.

3. The portable ultrasound cart of claim 2, wherein the release lever is positioned to allow a user to actuate the release lever and lift the portable ultrasound device off of the portable ultrasound cart with a single hand.

4. The portable ultrasound cart of claim 2, wherein the release lever is positioned to allow a user to automatically secure the portable ultrasound device to the portable ultrasound cart with a single hand.

5. The portable ultrasound cart of claim 1, wherein the planar surface defines grooves configured to receive feet of the portable ultrasound device and align the engagement features of the portable ultrasound device with the engagement features of the portable ultrasound cart.

6. The portable ultrasound cart of claim 5, wherein the grooves are configured to align the engagement features of the portable ultrasound device with the engagement features of the portable ultrasound cart such that the portable ultrasound device can be automatically secured to the portable ultrasound cart.

7. The portable ultrasound cart of claim 1, further comprising a control circuit configured to adjust the tilt angle of the support structure by controlling a tilt actuator of the tilt system based on at least one of a state of the portable ultrasound device, an identification of an operator, or a previously stored tilt angle.

8. The portable ultrasound cart of claim 7, further comprising a tilt control switch configured to provide an input to the control circuit, wherein the control circuit adjusts the tilt angle by controlling the tilt actuator in response to the received input from the tilt control switch.

9. The portable ultrasound cart of claim 7, wherein the control circuit automatically adjusts the tilt angle based on predefined preferences of the operator responsive to the identification of the operator, wherein the identification is based on at least one of an IP address, a serial number, or a user profile of the portable ultrasound device, and wherein identifying information is received from at least one of a transceiver of the portable ultrasound device, a facial recognition system, a radio frequency identification circuit, or a mobile communications device.

10. The portable ultrasound cart of claim 7, wherein the control circuit is configured to determine that the portable ultrasound device has been turned off or the portable ultrasound device has been disengaged from the support structure and in response to the determination controls the tilt actuator to lower the support structure to a rest position.

11. The portable ultrasound cart of claim 7, further comprising a plurality of user preference inputs trainable to correspond to user position preferences of different users, wherein when one of the plurality of user preference inputs is actuated, the control circuit adjusts the tilt angle by controlling the tilt actuator.

12. The portable ultrasound cart of claim 1, further comprising:
a transducer housing structured to receive an ultrasound transducer, the ultrasound transducer configured to provide data to the portable ultrasound device;
a rotation actuator coupled to the transducer housing and configured to rotate the transducer housing; and
a control circuit configured to provide a command to the rotation actuator to rotate the transducer housing to a specific orientation based on at least one of a state of the portable ultrasound device, an identification of an operator, or a previously stored orientation.

13. The portable ultrasound cart of claim 12, wherein the control circuit automatically adjusts the orientation of the transducer housing based on predefined preferences of the operator responsive to the identification of the operator, wherein the identification of the operator includes at least one of an IP address, a serial number, or a user profile of the portable ultrasound device.

14. The portable ultrasound cart of claim 12, wherein:
the transducer housing is configured to be positioned in a stored orientation and a non-stored orientation, wherein in the stored orientation at least a portion of the transducer housing is closer to a center position of the portable ultrasound cart than in the non-stored orientation; and
the control circuit is configured to cause the transducer housing to rotate to the stored orientation in response to at least one of:
(1) the portable ultrasound device being turned on; or
(2) the portable ultrasound device being disengaged from the portable ultrasound cart.

15. The portable ultrasound cart of claim 12, wherein the transducer housing is rotated to an operating orientation responsive to at least one of the portable ultrasound device being turned ON or the portable ultrasound device being coupled to the portable ultrasound cart.

16. The portable ultrasound cart of claim 1, further comprising;
a vertical support member including:
a first support member;
a second support member, wherein the first support member is positioned within the second support member providing a telescoping feature, the telescoping feature configured to variably adjust of a height of the portable ultrasound cart;
a height actuator configured to extend and retract the first support member with respect to the second support member; and
a control circuit configured to provide a command to the height actuator to extend or retract the first support member to a specific height based on at least one of a state of the portable ultrasound device, an identification of an operator, or a previously stored height.

17. The portable ultrasound cart of claim 16, wherein the control circuit automatically adjusts the height of the portable ultrasound cart based on a predefined preference of the operator responsive to the identification of the operator, wherein the identification is based on at least one of an IP address, a serial number, or a user profile of the portable ultrasound device, and wherein identifying information is received from at least one of a transceiver of the portable ultrasound device, a radio frequency identification circuit, or a mobile communications device.

18. The portable ultrasound cart of claim 16, wherein the control circuit automatically adjusts the height of the portable ultrasound cart to a stored height responsive to at least one of the state of the portable ultrasound device being turned off or the portable ultrasound device being disengaged from the support structure.

19. The portable ultrasound cart of claim 1, wherein the wall and the planar surface of the portable ultrasound cart are configured to receive a lower layer of the portable ultrasound device such that the wall obscures the lower layer when the portable ultrasound device is coupled to the portable ultrasound cart, and wherein a middle layer of the portable ultrasound device is flush with the wall of the portable ultrasound cart when the portable ultrasound device is coupled to the portable ultrasound cart.

20. The portable ultrasound cart of claim 1, further comprising a handrail positioned adjacent to a portion of the support structure at which the release lever is located, the handrail shaped to fit a hand of a user.

21. A portable ultrasound cart, comprising:
a thermal management system configured to enhance thermal management capabilities of a portable ultrasound device coupled to the portable ultrasound cart, the thermal management system including a fan configured to supplement an internal fan of the portable ultrasound device, the fan included in a support structure configured to receive the portable ultrasound device;
wherein the fan is positioned to increase airflow through a passageway between the portable ultrasound device and the portable ultrasound cart;
wherein the portable ultrasound device includes a bottom surface that defines a first plurality of slots and the portable ultrasound cart includes a planar surface that defines a second plurality of slots, wherein the first plurality of slots and the second plurality of slots substantially align when the portable ultrasound device and the portable ultrasound cart are coupled together.

22. The portable ultrasound cart of claim 21, wherein the fan is positioned upstream of the internal fan of the portable ultrasound device and configured to push ambient air into the portable ultrasound device through the passageway.

23. The portable ultrasound cart of claim 21, wherein the fan is positioned downstream of the internal fan of the portable ultrasound device and configured to pull heated air out the portable ultrasound device.

24. The portable ultrasound cart of claim 21, wherein the support structure includes one or more vents positioned on one or more sides of the support structure.

25. The portable ultrasound cart of claim 21, further comprising a second fan, wherein the fan is positioned upstream of the internal fan of the portable ultrasound device and configured to push ambient air into the portable ultrasound device through the passageway, and wherein the second fan is positioned downstream of the internal fan of the portable ultrasound device and configured to pull heated air out the portable ultrasound device.
